(19) United States
(12) Patent Application Publication
(10) Pub. No.: US 2026/0137701 A1
(43) Pub. Date: May 21, 2026

(54) CHOLESTENOIC ACID (CA) AND A SULFATED DERIVATIVE THEREOF, 3[BETA]-SULFATE-5-CHOLESTENOIC ACID (CA3S), AS ENDOGENOUS EPIGENETIC REGULATORS

(71) Applicant: VIRGINIA COMMONWEALTH UNIVERSITY, Richmond, VA (US)

(72) Inventors: Shunlin REN, Manakin-sabot, VA (US); Yaping WANG, Richmond, VA (US)

(21) Appl. No.: 19/118,972

(22) PCT Filed: Oct. 5, 2023
(86) PCT No.: PCT/US2023/034506

§ 371 Date: Apr. 7, 2025

Related U.S. Application Data
(60) Provisional application No. 63/413,641, filed on Oct. 6, 2022.
(60) Provisional application No. 63/431,456, filed on Dec. 9, 2022.
(60) Provisional application No. 63/460,742, filed on Apr. 20, 2023.
(60) Provisional application No. 63/471,563, filed on Jun. 7, 2023.
(60) Provisional application No. 63/532,726, filed on Aug. 15, 2023.

(51) Int. Cl. A61K 31/575; A61P 1/16; C07J 9/00

(52) U.S. Cl. CPC A61K 31/575; A61P 1/16; C07J 9/005

(57) ABSTRACT

3 β-hydroxy-5-cholestenoic acid 3-sulfate (CA3S), a sulfated derivative of 3β-hydroxy-5-cholestenoic acid (CA), is provided. Both CA and CA3S have potent cholesterol and triglyceride lowering and anti-inflammatory activities. Methods of using CA and CA3S to lower lipids and prevent or treat inflammation-associated diseases are also provided.

### CROSS-REFERENCE TO RELATED APPLICATIONS

This application claims benefit of U.S. provisional patent applications 63/413,641, filed Oct. 6, 2022; 63/431,456 filed Dec. 9, 2022; 63/460,742 filed Apr. 20, 2023; 63/471,563 filed Jun. 7, 2023; and 63/532,726 filed Aug. 15, 2023.

### STATEMENT OF FEDERALLY SPONSORED RESEARCH AND DEVELOPMENT

This invention was made with government support under Merit Review grant 1I01BX003656 awarded by the Veterans Administration. The United States government has certain rights in the invention.

### BACKGROUND OF THE INVENTION

### Technical Field

The invention generally relates to 3 β-hydroxy-5-cholestenoic acid 3-sulfate (CA3S), a sulfated derivative of 3β-hydroxy-5-cholestenoic acid (CA), and the potent cholesterol and triglyceride lowering and anti-inflammatory activities of both CA and CA3S. In particular, the invention provides methods of using CA and CA3S to prevent or treat lipid accumulation- and inflammation-associated diseases.

### Description of Related Art

There are two hepatic pathways for bile acid biosynthesis, “classic” and “acidic” pathways. The classic pathway is initiated by microsomal cholesterol 7-hydroxylase (CYP7A1) and followed by sterol 12-hydroxylation to produce cholic acid. The “acidic” pathway of bile acid biosynthesis is initiated by mitochondrial sterol 27-hydroxylase (CYP27A1) followed by 7α-hydroxylation by CYP7B1. Interestingly, CYP27A1 can synthesize 27-hydroxycholesterol (27HC), 25-hydroxycholesterol (25HC), and cholestenoic acid (CA), which have been shown to be naturally occurring ligands for Liver X Receptors (LXRs).

25HC and 27HC can be sulfated at the 3β-hydroxyl group and the sulfated forms counter effects of oxysterols by decreasing lipid biosynthesis, suppressing inflammatory responses, and promoting cell survival.

CA is the only primary bile acid which does not contain a 7-hydroxyl group. CA has been reported to be a biomarker of alveolar macrophages functional integrity and its cellular level decreases with increasing disease severity in patients with acute respiratory distress syndrome (ARDS). CA has also been reported to act as an endogenous γ-secretase modulator (GSM) within the brain and it has been hypothesized that increased levels of CA in the brain maybe help prevent Alzheimer's disease (AD). However, its activation of biochemical signaling pathway(s) and mechanism of action is currently unknown.

There is a need in the art for clarification of the biological role of CA and identification of biologically relevant modified forms thereof, and to determine useful methods of treating a variety of diseases using these compounds.

### SUMMARY OF THE INVENTION

The present disclosure provides 3β-hydroxy-5-cholestenoic acid 3-sulfate (CA3S3S), a novel sulfated derivative of 3β-Hydroxy-5-cholestenoic acid (CA). Both CA and CA3S have been found to have potent cholesterol and triglyceride lowering and anti-inflammatory properties. Accordingly, methods of using CA and CA3S to treat a variety of lipid accumulation and inflammation-related diseases are also provided.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

It is an object of this invention to provide a compound which is 3 β-hydroxy-5-cholestenoic acid 3-sulfate (CA3S) or salts or solvates thereof.

It is a further object of the invention to provide a pharmaceutical composition comprising the compound of claim 1 (CA3S or salts or solvates thereof) and a pharmaceutically acceptable carrier, the compound being dissolved or distributed in the carrier, wherein the compound comprises 1-99% of the composition. In some aspects, the pharmaceutical composition is formulated in unit dosage form. In other aspects, the composition is in solid form. In additional aspects, the composition is in the form of a powder, a tablet, a capsule or a lozenge; or the composition comprises the compound in freeze-dried form together with a bulking agent. In other aspects, the carrier is a liquid. In yet further aspects, the compound is solubilized in the liquid or dispersed in the liquid; and/or the liquid is aqueous; and/or the liquid is sterile water for injections or phosphate-buffered saline; and/or the composition is in a sealed vial, ampoule, syringe or bag.

Also provided is a method of treating a subject, which method comprises administration to the subject of an effective amount of a compound as defined in claim 1, wherein the method is selected from: a method for treating sepsis in a subject in need thereof; a method for treating metabolic associated fatty liver diseases in a subject in need thereof; a method for reducing lipids in a subject in need thereof; a method of reducing cholesterol and lipid biosynthesis in a subject in need thereof; a method of reducing inflammation in a subject in need thereof; a method of treating diabetes in a subject in need thereof; a method of treating hyperlipidemia in a subject in need thereof; a method of treating atherosclerosis in a subject in need thereof; a method of treating fatty liver disease in a subject in need thereof; and a method of treating inflammatory disease in a subject in need thereof. In some aspects, the compound is administered in an amount ranging from 0.1 mg/kg to 100 mg/kg based on body mass of the subject, or the compound is administered in an amount ranging from 1 mg/kg to 10 mg/kg, based on body mass of the subject; and/or the administration comprises at least one of oral administration, enteric administration, sublingual administration, transdermal administration, intravenous administration, peritoneal administration, parenteral administration, administration by injection, subcutaneous injection, and intramuscular injection.

Also provided is a method of lowering lipids in a subject in need thereof, comprising administering to the subject a therapeutically effective amount of at least one of 3β-hydroxy-5-cholestenoic acid (CA) and CA3S. In some aspects, the lipids are cholesterol and/or triglycerides. In further aspects, the therapeutically effective amount is from 1-100 mg/kg of body weight.

Also provided is a method of preventing or treating an inflammatory disease or condition in a subject in need thereof, comprising administering to the subject a therapeutically effective amount of at least one of CA and CA3S. In some aspects, the inflammatory disease or condition is sepsis, metabolic associated fatty liver diseases, or atherosclerosis. In other aspects, the therapeutically effective amount is from 1-100 mg/kg of body weight.

Also provided is a method of preventing or treating non-alcoholic fatty liver disease (NAFLD) in a subject in need thereof, comprising administering to the subject a therapeutically effective amount of at least one of CA and CA3S. In some aspects, the therapeutically effective amount is from 1-100 mg/kg of body weight.

Also provided is a method of treating cancer in a subject in need thereof, comprising administering to the subject a therapeutically effective amount of CA. In some aspects, the cancer is hepatocellular carcinoma.

### BRIEF DESCRIPTION OF THE DRAWINGS

FIG. 1A-G. Biosynthesis and enzyme kinetic study of CA. Panel A: The biosynthesis of 25HC, 27HC, and CA in mitochondria. B: Concentration (0-0.0001 M)-dependent effect of 25HC and 27HC, on DNMT1/DNMT3a/DNMT3b enzymatic activity. C: Effects of CA on DNMT1 enzymatic activity. D: Effects of CA on DNMT3a enzymatic activity. E: Effects of CA on DNMT3b enzymatic activity. Effects of CA on DNMT1, EC50=9.13±0.66×10-6 M (C. mean±SD; n=3); DNMT3a, IC50=8.41±4.53×10-6 M (D. mean±SD; n=3); DNMT3b, IC50=4.89±1.82×10-6 M (E. mean±SD; n=3). F: Serum activities in NAFLD mouse models. The mice were treated with 10 mg/kg CA injection for 2 weeks. The sera were collected and the activities of ALT, AST, and ALK were determined by a clinical laboratory. Control represents control mice with DMSO injection only.

FIGS. 2A and B. LC-MS/MS analysis of CA levels following CA treatment of HepG-2 cells, time course. A: CA levels in total cellular fractions; B: CA levels in nuclear fractions.

FIGS. 3A and B. Western blot analysis of AMPK protein expression in the cells following CA treatment for 24 hours. HepG-2 cells were cultured in high glucose media for 72 hours and then treated with 20 μM of CA for 24 hours. Total proteins were extracted from the treated cells. The extracted proteins, 20 ug, were separated by SDS-PAGE analysis. The specific AMPK protein was identified by Western Blot analysis. A, SDS-PAGE; B, fold change in relative band intensity.

FIG. 4A-D. Effects of CA on DNA methylation in hepatocytes using whole genome bisulfite sequencing (WGBS) analysis. HepG-2 cells were cultured in HG medium for 72 hours and followed by treatment with 20 μM CA treatment for 0, 3, 6, 12, and 24 hours. One μg of genomic DNA was used to prepare libraries. Panel A: Number of differential methylated regions (DMRs) in whole genome. B: Number of DMRs in promoter regions. C: Top terms of Gene Ontology (GO) analysis, enriched in hypomethylated DMRs in promoter regions. LMP: lipid metabolic process; PRE: positive regulation of ERK1 and ERK2 cascade; CMP: carbohydrate metabolic process; PRM: positive regulation of MAPK cascade; LCP: lipid catabolic process; FAM: fatty acid metabolic process; TCC: tricarboxylic acid cycle; NRC: negative regulation of cell growth; PRI: positive regulation of interleukin-6 production; MIM: mitochondrial inner membrane; MME: mitochondrial membrane; MOM: mitochondrial outer membrane; MMA: mitochondrial matrix; ERM: endoplasmic reticulum membrane; EEX: extracellular exosome; ADI: Z disc; ICM: integral component of mitochondrial inner membrane; MMB: membrane; ESP: extracellular space; LDA: L-lactate dehydrogenase activity; AEA: 1-alkyl-2-acetylglycerophosphocholine esterase activity; EAA: enzyme activator activity; PKC: protein kinase A catalytic subunit binding; PSA: protein self-association; SPB: S100 protein binding; CEA: cysteine-type endopeptidase activator activity involved in apoptotic process; UPL: ubiquitin protein ligase binding; GRB: GABA receptor binding; CCR: complement component C5a receptor activity. D: Top enriched KEGG pathways of promoter region with hypomethylated DMRs.

FIG. 5A-G. Effect of CA on transcriptional activities in hepatocytes. HepG-2 cells were cultured in HG medium and treated with 20 μM of CA for 0, 3, 6, 12, and 24 hours. Panel A: The number of down-regulated genes regulated by CA. B: The number of up-regulated genes by CA. C: Top GO terms that enriched by down regulated genes treated by 20 μM CA for 6 hours. CBP: cholesterol biosynthetic process; IBP: isoprenoid biosynthetic process; SBP: sterol biosynthetic process; SDBP: steroid biosynthetic process; IDBP: isopentenyl diphosphate biosynthetic process, mevalonate pathway; CI: cholesterol import; RN: response to nutrient; CMP: cholesterol metabolic process; CH: cholesterol homeostasis; NRLLPC: negative regulation of low-density lipoprotein particle clearance. D: Top GO terms that were enriched by up regulated genes following treatment with 20 μM CA for 6 hours. CRCI: cellular response to copper ion; CZIH: cellular zinc ion homeostasis; DCI: detoxification of copper ion; NRG: negative regulation of growth; CRZI: cellular response to zinc ion; CRCI: cellular response to cadmium ion; AMP: ATP metabolic process; CRE: cellular response to erythropoietin; ACO: actin cytoskeleton organization; PLAJ: protein localization to adherens junction. E: KEGG pathways enriched by down regulated genes treated by 20 μM CA for 6 hours, involved gene numbers were labeled at the end of each bar. F: The gene-gene network analysis revealed that the down-regulated genes are involved in KEGG pathways. G: Heatmap for the expression levels of down-regulated genes that enriched in cholesterol metabolism, metabolic pathways, and steroid biosynthesis pathways. PCSK9: Proprotein convertase subtilisin/kexin type 9; MVK: Mevalonate Kinase; HMGCS1: 3-Hydroxy-3-Methylglutaryl-CoA Synthase 1; MVD: Mevalonate Diphosphate Decarboxylase; MSMO1: Methylsterol Monooxygenase 1; IDI1: Isopentenyl-Diphosphate Delta Isomerase 1; HMGCR: 3-Hydroxy-3-Methylglutaryl-CoA Reductase; FDFT1: Farnesyl-Diphosphate Farnesyltransferase 1; CYP51A1: Cytochrome P450 Family 51 Subfamily A Member 1; HSD17B7: Hydroxysteroid 17-Beta Dehydrogenase 7.

FIGS. 6Aand B. RT-qPCR analysis of gene expression involved in calcium signaling and lipids metabolism pathways. HepG-2 cells were cultured in HG medium for 72 hours, followed by treating with 0, 2.5, 5, 10, and 20 μM CA treatment for 6 hours, and 20 μM CA treatment for 0, 3, 6, 12, and 24 hours. The expression of key genes involved in calcium signaling and lipids metabolism pathways were measured by RT-qPCR. Panel A: Dose and time dependent expression of key genes involved in lipid metabolism signaling pathway. B: Dose and time dependent expression of key genes involved in calcium signaling pathway.

FIG. 7A-F. Effect of CA on lipid accumulation in hepatocytes. HepG-2 cells were cultured in HG medium for 72 hours, followed by treatment of 20 μM CA for another 24, 48, and 72 hours. The lipids levels were measured by untargeted lipidomics assay. Panel A: the total lipids relative re-sponse of CA vs vehicle treatment at 48 hours. B: top decreased ChE (cholesterol ester) lipidlon. C: top decreased FA (fatty acid) lipidlon. D: top decreased MG (Monoglycerides) lipidlon. E: top decreased DG (Diglycerides) lipidlon. F: top decreased TG (Triglycerides) lipidlon.

FIG. 8. Proposed model of CA gene regulation in hepatocytes. A high sugar diet produces an excess of acetyl-CoA, which can be used to synthesize cholesterol and long chain fatty acids. Cholesterol is a precursor for the synthesis for 25HC, 27HC, and CA in mitochondria. These oxysterols bind LXRs for transport to the nucleus where they regulate genes involved in calcium-AMPK and fatty acid and cholesterol biosynthetic pathways. 25HC, 27HC, and CA may play different roles in the pathophysiology of NAFLD. The dashed red lines represent known pathways, and the blue solid lines represent the proposed pathways regulated by CA.

FIG. 9A-C. Hepatocellular carcinoma (HCC). A, disease progression; B, methylation levels in HCC; C, cholestenoic acid (CA) significantly inhibits DNA methyltransferases 1/3a/3b (DNMT 1/3a/3b).

FIGS. 10A and B. CA induces HepG-2 cell death (A) but not the death of normal primary human hepatocytes (B).

FIG. 11A-C. Serum enzymatic activities of A, alkaline phosphatase (ALK), B, alanine aminotransferase (ALT), and C, aspartate aminotransferase (AST) in control mice vs mice treated with CA.

FIG. 12A-F shows the 3b-hydroxy cholestenoic acid (M/Z=415) decreases and 3b-sulfate cholestenoic acid increases in hepatocytes during culturing at 0 (A), 1.5 (B), 3 (C), 6 (D), and 24 (E) hrs. Incubation time is shown in F.

FIGS. 13A and B. A, MS-MS results of identification of 3β-sulfate, 5-cholestenoic acid; B, HPLC results of chemically synthesized CA3S.

FIG. 14. Endogenous cholestenoic acid derivative, 3β-sulfate-5-cholestenoic acid (CA3S) suppresses key gene expressions involved in LPS induced inflammatory responses in macrophages.

FIG. 15A-C. Serum enzymatic activities of A, alkaline phosphatase (ALK), B, alanine aminotransferase (ALT), and C, aspartate aminotransferase (AST) in control mice vs mice treated with CA3S.

### DETAILED DESCRIPTION

As shown in the Examples presented herein, CA is a mitochondrial cholesterol metabolite that decreases cholesterol and triglyceride biosynthesis by suppressing gene expression of key genes involved in the lipid biosynthesis, leading to lower levels of intracellular and serum cholesterols and triglycerides. Further, CA has potent anti-inflammatory properties and anti-cancer activity. Thus, methods of using CA to prevent (e.g., prophylactically treat) or treat diseases associated with (caused by or related to) elevated cholesterol and triglycerides and/or inflammation, including cancer, are described.

In addition, as shown in the Examples presented herein, CA is also able to block cell apoptosis by increasing gene expression involved in anti-apoptosis and cell survival. Thus, methods of using CA to treat or prophylactically treat diseases and conditions associated with (caused by or related to) unwanted or abnormal apoptotic activity, thereby promoting the recovery from diseases and/or conditions caused by or associated with injured cells and/or organs.

Accordingly, in some aspects, provided herein are methods of using 3β-hydroxy-5-cholestenoic acid (CA) having the following chemical formula

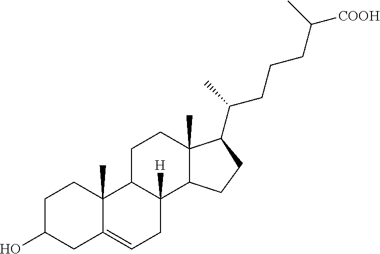

and salt forms thereof, to prevent and/or treat a variety of diseases and conditions as described herein.

In other aspects, the present invention provides a novel derivative of CA, 3 β-hydroxy-5-cholestenoic acid 3-sulfate (CA3S), having the formula

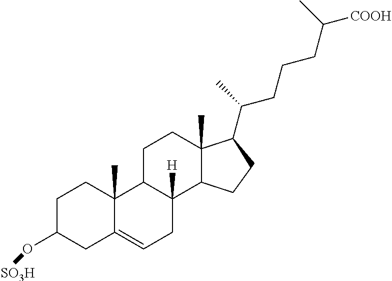

and salt forms thereof. CA3S is derivative of cholestenoic acid which is secreted from hepatocytes and acts on macrophages. CA3S has potent cholesterol and triglyceride lowering and anti-inflammatory properties. CA3S suppresses inflammatory responses by suppressing pro-inflammation gene expression. The decreases in pro-inflammation cytokine gene expression advantageously lead to suppression of unwanted or abnormal inflammatory responses. Thus, CA3S is useful for treating diseases associated with inflammatory responses, such as sepsis, metabolic associated fatty liver diseases, and atherosclerosis. This disclosure provides both the compound and uses thereof for treating a variety of diseases and conditions as described herein.

### Forms of CA3S And CA

The CA and CA3S described herein are natural products. However, in some aspects, they are provided or used in the methods described herein in forms that are not natural, biological forms.

For example, in some aspects, the CA3S and CA are isolated. “Isolated” means not comprised within tissue material contained within, or extracted from, a human or animal subject. For example, an isolated compound is not comprised within a cell. Thus, isolated CA3S or CA is clearly distinguishable from native CA3S or CA that is comprised within tissue material (e.g., a cell) that is itself contained within, or has been extracted from, a human or animal subject.

In some aspects, the CA3S and CA are substantially pure. When it is “substantially pure” or “substantially purified” the compounds are in a form that is at least about 75%, preferably at least about 80%, more preferably at least about 90%, and most preferably at least about 95% or more (e.g., 96, 97, 98 or 99%) free from other chemical species. Substantially pure CA and/or CA3S may, in particular, comprise at least about 90 wt % or at least about 95%, and more preferably at least about 98 wt %, at least about 99 wt % or, even more preferably, at least about 99.5 wt % or at least about 99.8 wt % of CA and/or CA3S.

In some aspects, the CA and CA3S are in the form of substantially purified salts, especially pharmaceutically acceptable salts such as any of the relatively non-toxic, inorganic and organic acid addition salts and base addition salts discussed elsewhere herein (see, for example, the “Pharmaceutical Compositions” section below). The substantially purified salt may be a solid, such as a crystalline solid.

In some aspects, the CA and CA3S (and/or salts thereof) are in solid form. Those of skill in the art are aware that a solid is one of the four fundamental states of matter (along with liquid, gas, and plasma). The molecules of a solid are closely packed together and contain the least amount of kinetic energy, compared to the other states of matter. A solid is characterized by structural rigidity and resistance to a force applied to the surface. When in solid form, the solid generally comprises a plurality of CA or CA3S molecules. The solid may be crystalline or amorphous or a mixture of both. The CA and/or the CA3S may be in the form of particles, nano-particles, sheets, solid three-dimensional objects (e.g., a sphere, an ovoid, a rectangle, a block, an irregularly-shaped solid, etc.), where a plurality of CA and/or CA3S molecules (or salts thereof) are associated with each other. Generally, the solid comprises or consists of only CA or CA3S (or mixtures and/or salts thereof) that are packed together amorphously or in a crystalline form, or a combination of both, separated by atomic level spacing (i.e., the distance between the nuclei of the atoms which make up the molecule). In solid form, the nuclei of atoms that make up a compound are generally separated from each other by only a few angstroms. None of these solid forms are found in nature, where CA and/or CA3S molecules are generally spaced apart in a fluid and/or membrane (e.g., an aqueous or lipid milieu) or attached to other biological molecules.

In some aspects, the CA or the CA3S are in an artificial (non-natural) liquid or solid composition such as in a buffered solution (e.g. a working solution for storage during or after isolation or synthesis), a composition used for analytic purposes (e.g., in a laboratory), a pharmaceutical composition suitable for administration to a subject, etc., none of which are found in nature. In such compositions, the concentration of CA or CA3S is greater than is found in nature.

### Compositions

The present disclosure encompasses compositions (formulations) such as pharmaceutical compositions, comprising at least one of CA and CA3S, and the description provided here applies to both compounds. The compositions generally include one or more substantially purified compounds or salts of at least one of the compounds as described herein, and a pharmacologically suitable (physiologically compatible) carrier. In some aspects, such compositions are prepared as liquid solutions or suspensions, or as solid forms such as tablets, pills, powders and the like. Solid forms suitable for solution in, or suspension in, liquids prior to administration are also contemplated (e.g., lyophilized forms of the compounds), as are emulsified preparations. In some aspects, the formulations are liquid and are aqueous or oil-based suspensions or solutions.

In some aspects, the active ingredients (e.g., CA and/or CS) are mixed with excipients and/or carriers which are pharmaceutically acceptable and compatible with the active ingredients. Suitable excipients include, for example, water, saline, dextrose, glycerol, ethanol and the like, or combinations thereof. In addition, the composition may contain minor amounts of auxiliary substances such as wetting or emulsifying agents, pH buffering agents, preservatives, and the like. If it is desired to administer an oral form of the composition, various thickeners, flavorings, diluents, emulsifiers, dispersing aids or binders and the like are added. The composition of the present invention may contain any such additional ingredients so as to provide the composition in a form suitable for administration.

Depending on the formulation, it is expected that the active agent (CA or CA3S) will make up about 1% to about 99% by weight of the composition and the vehicular “carrier” will constitute about 1% to about 99% by weight of the composition. The pharmaceutical compositions of the present invention may include any suitable pharmaceutically acceptable additives or adjuncts to the extent that they do not hinder or interfere with the therapeutic effect of the CA or CA3S. Still other suitable formulations for use in the present invention are found, for example in Remington's Pharmaceutical Sciences, 22nd ed. (2012; eds. Allen, Adejarem Desselle and Felton).

Some examples of materials which can serve as pharmaceutically acceptable carriers include, but are not limited to: ion exchangers, alumina, aluminum stearate, lecithin, serum proteins (such as human serum albumin), buffer substances (such as Tween® 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; one or more cyclodextrins; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In some aspects, “pharmaceutically acceptable salts” of the compounds refers to the relatively non-toxic, inorganic and organic acid addition salts and base addition salts of compounds of the present disclosure. In some aspects, these salts are prepared in situ during the final isolation and purification of the compounds. In particular, acid addition salts can be prepared by separately reacting a purified compound in a free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting a purified compound in its acid form with a suitable organic or inorganic base and isolating the salt thus formed. Base addition salts include pharmaceutically acceptable metal and amine salts. Suitable metal salts include sodium, potassium, calcium, barium, zinc, magnesium, and aluminum salts. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like. In some aspects, the pharmaceutically acceptable salt is, for example, an alkali metal salt (e.g., a lithium, sodium or potassium salt), an alkaline earth metal salt (e.g., a calcium salt) or an ammonium salt. The pharmaceutically acceptable salt may, for example, be a sodium, potassium, calcium, lithium, hydrochloride or ammonium salt.

The compounds may also be formulated for modified release. Modified-release dosage is a mechanism that (in contrast to immediate-release dosage) delivers a drug with a delay after its administration (delayed-release dosage) or for a prolonged period of time (extended-release [ER, XR, XL] dosage) or to a specific target in the body (targeted-release dosage). Such formulations provide a delayed, long-acting, extended and/or sustained release of the CA or CA3S. Sustained-release dosage forms are dosage forms designed to release (liberate) a drug at a predetermined rate to maintain a constant drug concentration for a specific period of time with minimum side effects. This can be achieved through a variety of formulations, including liposomes and drug-polymer conjugates (an example being hydrogels). Sustained release's definition is more akin to a “controlled release” rather than “sustained”. Extended-release dosage consists of either sustained-release (SR) or controlled-release (CR) dosage. SR maintains drug release over a sustained period but not at a constant rate. CR maintains drug release over a sustained period at a nearly constant rate. All such formulations include the dosage forms described herein and/or any form of “implant”, i.e., implantable devices, pellets, depots, etc.

In some aspects, the compositions are pharmaceutical compositions which are formulated in unit dosage form. In some aspects, the pharmaceutical composition is in solid form, including but not limited to: a powder, a tablet, a capsule or a lozenge; or the composition comprises the compound in freeze-dried form together with a bulking agent, the composition optionally being in a sealed vial, ampoule, syringe or bag. For example, the CA, the CA3S, or the pharmaceutically acceptable salt thereof may be in the form of a powder or a freeze-dried form. As is well-known, freeze-drying is a dehydration process typically used to preserve perishable material or make the material more convenient for transport. There are three main stages to this technique, namely freezing, primary drying and secondary drying. Freezing is typically performed using a freeze-drying machine. During primary drying the pressure is controlled by the application of appropriate levels of vacuum whilst enough heat is supplied to enable any water present to sublimate. In the secondary drying process, water of hydration is removed by the further application of heat. Typically, the pressure is also lowered to encourage further drying. After completion of the freeze-drying process, the vacuum can either be broken with an inert gas such as nitrogen prior to sealing or the material can be sealed under vacuum.

In some aspects, the pharmaceutical composition comprises a carrier that is a liquid, for example, an aqueous buffer. Suitable aqueous buffers include, but are not limited to, acetate, succinate, citrate, and phosphate buffers varying in strength from 5 mM to 100 mM. In some cases, the aqueous buffer includes reagents that provide an isotonic solution. Such reagents include, but are not limited to, sodium chloride, and sugars, e.g., mannitol, dextrose, sucrose, and the like. In some cases, the aqueous buffer further includes a non-ionic surfactant such as polysorbate 20 or 80. In some instances, compositions of interest further include a preservative. Suitable preservatives include, but are not limited to, benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In this aspect, the compound may be solubilized in the liquid or dispersed in the liquid; and/or the liquid is aqueous; and/or the liquid is sterile water for injections or phosphate-buffered saline; and/or the composition is in a sealed vial, ampoule, syringe or bag.

CA and/or CA3S may be administered in pure form or in a pharmaceutically acceptable formulation. Such formulations (compositions) typically include CA or CA3S or a pharmaceutically acceptable salt thereof and a physiologically acceptable (compatible) excipient, diluent or carrier/vehicle. The CA or CA3S may be, for example, in the form of a pharmaceutically acceptable salt (e.g., an alkali metal salt such as sodium, potassium, calcium, lithium, ammonium, etc.), or other complex.

The pharmaceutical composition is sterile. Sterile means substantially free of viable microbes, for example as determined using the USP sterility test (see “The United States Pharmacopeia”, 30th Revision, The United States Pharmacopeial Convention: 2008.). To maintain sterility, the pharmaceutical composition may be presented in a sealed package that is capable of preventing ingress of viable microbes. For example, in the case of a liquid pharmaceutical composition, the composition may be sterilized and sealed in a vial or ampoule.

It should be understood that pharmaceutically acceptable formulations (compositions) include liquid and solid materials conventionally utilized to prepare both injectable dosage forms and solid dosage forms such as tablets, lozenges, powders and capsules, as well as aerosolized dosage forms. The compounds may be formulated with aqueous or oil-based vehicles. Water may be used as the carrier for the preparation of compositions (e.g., injectable compositions), which may also include conventional buffers and agents to render the composition isotonic and to maintain a physiologically acceptable pH. Other potential additives (preferably those which are generally regarded as safe [GRAS]) include: colorants; flavorings; surfactants (TWEEN™, oleic acid, etc.); solvents, stabilizers, elixirs, and binders or encapsulants (lactose, liposomes, etc.). Solid diluents and excipients include lactose, starch, conventional disintegrating agents, coatings and the like. Preservatives such as methyl paraben or benzalkium chloride may also be used.

In further detail, when the composition is in solid form it may be in the form of a powder, a tablet, a capsule or a lozenge. When the composition is in solid form the composition may comprise the CA or CA3S in freeze-dried form together with a bulking agent. A bulking agent is a pharmaceutically inactive and typically chemically inert substance that may be added to a composition to increase its bulk. Common bulking agents for use in the preparation of freeze-dried pharmaceutical compositions, and which are suitable here, include mannitol and glycine. When the composition is in solid form it may optionally be in a sealed vial, ampoule, syringe or bag.

When the pharmaceutical composition comprises a liquid carrier, the CA or CA3S may be solubilized in a liquid or dispersed in a liquid; and/or the liquid may be aqueous; and/or the liquid may be sterile water for injections or phosphate-buffered saline. When the pharmaceutical composition comprises a liquid carrier, the composition may be in a sealed vial, ampoule, syringe or bag.

### Administration

The pharmaceutical compositions disclosed herein are administered in vivo by any suitable route including but not limited to: inoculation or injection (e.g. intravenous, intraperitoneal, intramuscular, subcutaneous, intra-aural, intraarticular, intramammary, and the like), topical application (e.g. on areas such as eyes, skin, in ears or on afflictions such as wounds and burns) and by absorption through epithelial or mucocutaneous linings (e.g., nasal, oral, vaginal, rectal, gastrointestinal mucosa, and the like). Other suitable means include but are not limited to: inhalation (e.g. as a mist or spray), orally (e.g. as a pill, capsule, liquid, etc.), intravaginally, intranasally, rectally, by ingestion of a food or probiotic or nutritional product containing the compound, as eye drops, incorporated into dressings or bandages (e.g. lyophilized forms may be included directly in a dressing), via a port, etc. In further detail, administration may be at least one of oral administration, enteric administration, sublingual administration, transdermal administration, intravenous administration, peritoneal administration, parenteral administration, administration by injection, subcutaneous injection, and intramuscular injection. For example, administration may be oral or parenteral, including intravenously, intramuscularly, subcutaneously, intradermal injection, intraperitoneal injection, etc., or by other routes (e.g., transdermal, sublingual, oral, rectal and buccal delivery, inhalation of an aerosol, etc.). In a preferred embodiment, administration is oral or by injection.

Further, administration of the compound may be carried out as a single mode of therapy, or in conjunction with other therapies, e.g., with other lipid or cholesterol lowering drugs, pain medications, exercise and diet regimens, surgery when warranted, organ transplant, etc. In the case of treating cancer, the compounds may be administered, for example, with other anticancer agents or therapies such as those listed in issued U.S. patent Ser. No. 11/433,106, surgery and/or radiation therapy. The administration of CA or CA3S to a patient may be intermittent, or at a gradual or continuous, constant or controlled rate. In addition, the time of day and the number of times per day that the pharmaceutical formulation is administered may vary and are best determined by a skilled practitioner such as a physician. Likewise, the duration of the treatment may vary and can be adjusted to accommodate the needs of the patient.

The exact dosage of CA or CA3S to be administered may vary depending on the age, gender, weight, overall health status of the individual patient, etc., as well as on the precise etiology of the disease. However, in general for administration in mammals (e.g. humans), therapeutically effective dosages are in the range of from about 0.1 to about 500 mg or more of compound per kg of body weight per 24 hr. (e.g., about 0.1 to about 25, 50, 100, 150, 200, 250, 300, 350, 400, 450, or 500 mg or more (e.g., up to about 600, 700, 800, 900 or even 1000 mg) of compound per kg of body weight per 24 hr. Typical doses range, for example, are from about 5, 25, 50, 75, 100, 125, 150, 175, 200, 225, 250, 275, 300, 325, 350, 400, 425, 450, or 500 mg of compound per kg of body weight per 24 hr (including all intervening integers) and frequently about 1 to about 100 mg of compound per kg of body weight per 24 hr., are effective.

In some aspects, the compound is administered in an amount ranging from 0.1 mg/kg to 100 mg/kg based on body mass of the subject, or the compound is administered in an amount ranging from 1 mg/kg to 10 mg/kg, based on body mass of the subject; and/or the administration comprises at least one of oral administration, enteric administration, sublingual administration, transdermal administration, intravenous administration, peritoneal administration, parenteral administration, administration by injection, subcutaneous injection and intramuscular injection.

As used herein a “therapeutically effective dose” is a dose that lessens (ameliorates) or eliminates at least one symptom of a disease or condition. While an optimal outcome may be the complete eradication of all symptoms (a “cure”), much benefit can accrue if only a few symptoms are completely eradicated, or if overall one or more symptoms is decreased, made less serious or less painful, life span is lengthened, the disease goes into remission, etc., even if all symptoms are not fully addressed.

A pharmaceutical composition of the invention may be formulated in unit dosage form, i.e., the pharmaceutical composition may be in the form of discrete portions each containing a unit dose of the CA or CA3S. In this context, a unit dose may comprise, for example, from about 0.1 mg to about 500 mg, or from about 0.5 mg to about 100 mg, or from about 1 mg to about 50 mg of CA or CA3S, or from about 5 mg to about 100 mg of CA or CA3S, including all integers in between these values. The pharmaceutical composition may be prepared by combining the CA or CA3S with the chosen physiologically acceptable excipients, diluents and/or carriers.

While the subjects are usually humans, veterinary applications of the technology are also contemplated.

### Methods and Uses

In further aspects, the invention provides methods of treating a subject (patient), which methods comprise administering to a subject in need thereof a therapeutically effective amount CA and/or CA3S. When the methods are practiced, one result is that the detectable, measurable level (amount, concentration) of CA or CA3S in the treated subject (for example, in a blood or plasma or biopsy sample) is greater than a comparable control level or range of levels. Those of skill in the art are familiar with the concept of determining suitable control levels or ranges. Such levels or ranges are typically determined by measuring the level of a substance of interest (e.g., CA or CA3S) in a statistically significant number of healthy “normal” subjects who have not been treated, and/or in a statistically significant number of subjects having the same disease or condition who have not been treated and/or in a statistically significant number of subjects having the same disease or condition who have been treated, for comparison. The determination of such levels or values is well within the skill of those in art.

The methods of treating generally involve identifying (e.g., diagnosing) a subject in need of the therapy, e.g., a subject or patient already suffering from at least one symptom of a malady, or at risk of suffering from at least one symptom of a malady (e.g., by virtue of a genetic predisposition, a disposition based on age, or by an impending procedure such as surgery, or for any other reason, etc.). The following sections describe diseases and conditions that can be treated by CA and/or CA3S. Those of skill in the art will recognize that the categories are not exclusive in that, for example, high lipid values are frequently accompanied by or exacerbated by inflammation.

In certain aspects, the method is selected from: a method for reducing lipids in a subject in need thereof; a method of reducing cholesterol and lipid biosynthesis in a subject in need thereof; a method of reducing inflammation in a subject in need thereof; a method of treating diabetes in a subject in need thereof; a method of treating hyperlipidemia in a subject in need thereof; a method of treating atherosclerosis in a subject in need thereof; a method of treating fatty liver disease in a subject in need thereof; and a method of treating inflammatory disease in a subject in need thereof.

### Reduction of Lipids and Treatment of Hyperlipidemia

Both CA and CA3S are used in methods to reduce (decrease) lipid levels in subjects in need thereof. In some aspects, the methods are directed to preventing or treating diseases and conditions caused, associated with or exacerbated by elevated lipid levels. In some embodiments, the disease or condition that is prevented or treated is or is caused by hyperlipidemia. By “hyperlipidemia” we mean a condition of abnormally elevated levels of any or all lipids and/or lipoproteins in the blood. Hyperlipidemia includes both primary and secondary subtypes, with primary hyperlipidemia usually being due to genetic causes (such as a mutation in a receptor protein), and secondary hyperlipidemia arising from other underlying causes such as diabetes (type I or type II). Lipids and lipid composites that may be elevated in a subject and lowered by the treatments described herein include but are not limited to chylomicrons, very low-density lipoproteins, intermediate-density lipoproteins, low-density lipoproteins (LDLs) and high-density lipoproteins (HDLs). In particular, elevated cholesterol (hypercholesteremia) and triglycerides (hypertriglyceridemia) are known to be risk factors for blood vessel and cardiovascular disease due to their influence on atherosclerosis. Lipid elevation may also predispose a subject to other conditions such as acute pancreatitis. The methods of the invention thus may also be used in the treatment or prophylaxis (e.g., prophylactic treatment) of conditions that are or are associated with elevated lipids. Such conditions include, for example, but are not limited to: hyperlipidemia, hypercholesterolemia, hypertriglyceridemia, metabolic syndrome, cardiovascular diseases, coronary heart disease, atherosclerosis (i.e. arteriosclerotic vascular disease or ASVD) and associated maladies, acute pancreatitis, various metabolic disorders, such as insulin resistance syndrome, diabetes, polycystic ovary syndrome, fatty liver disease (hepatic steatosis), cachexia, obesity, stroke, gall stones, inflammatory bowel disease, inherited metabolic disorders such as lipid storage disorders, and the like. In addition, various conditions associated with hyperlipidemia include those described in issued U.S. Pat. No. 8,003,795 (Liu, et al) and U.S. Pat. No. 8,044,243 (Sharma, et al), the complete contents of both of which are herein incorporated by reference in entirety.

### Inflammation

Both CA and CA3S are used in methods to prevent or treat disease and conditions involving excess or unwanted inflammation. However, CA3S is preferred for this purpose. The diseases and conditions that are prevented or treated include inflammation, and/or diseases and conditions associated with, characterized by or caused by inflammation. These include a large group of disorders which underlie many human diseases. In some embodiments, the inflammation is acute, resulting from e.g., an infection, an injury, etc. In other embodiments, the inflammation is chronic. In some embodiments, the immune system is involved with the inflammatory disorder as seen in both allergic reactions and some myopathies. However, various non-immune diseases with etiological origins in inflammatory processes may also be treated, including cancer, atherosclerosis, and ischemic heart disease, as well as others listed below.

Examples of disorders associated with abnormal inflammation which may be prevented or treated using CA and/or CA3S include but are not limited to: acne vulgaris, asthma, various autoimmune diseases, Celiac disease, chronic prostatitis, glomerulonephritis, various hypersensitivities, inflammatory bowel diseases, pelvic inflammatory disease, reperfusion injury, rheumatoid arthritis, sarcoidosis, transplant rejection, vasculitis, and interstitial cystitis. Also included are inflammation disorders that occur as a result of the use of both legally prescribed and illicit drugs, as well as inflammation triggered by negative cognitions or the consequences thereof, e.g., caused by stress, violence, or deprivation; sepsis and/or septicemia, and various metabolic associated fatty liver diseases (lipotoxicity).

In one aspect, the inflammatory disorder that is prevented or treated is an inflammatory bowel disease (IBD). There are three forms of IBD: Crohn's disease, ulcerative colitis, and indeterminate colitis, with differences mainly in the areas affected and the likely depth of inflammation. Any of these and other IBDs and be treated or prevented by administration of one or both of CA can CA3S.

In one aspect, the inflammatory disorder that is prevented or treated is an allergic reaction (type 1 hypersensitivity), the result of an inappropriate immune response that triggers inflammation. A common example is hay fever, which is caused by a hypersensitive response by skin mast cells to allergens. Severe inflammatory responses may mature into a systemic response known as anaphylaxis. Other hypersensitivity reactions (type 2 and type 3) are mediated by antibody reactions and induce inflammation by attracting leukocytes which damage surrounding tissue and may also be treated as described herein.

In other aspects, inflammatory myopathies are prevented or treated. Such myopathies are caused by the immune system inappropriately attacking components of muscle, leading to signs of muscle inflammation. They may occur in conjunction with other immune disorders, such as systemic sclerosis, and include dermatomyositis, polymyositis, and inclusion body myositis.

In one aspect, the methods and compositions of the invention are used to prevent or treat systemic inflammation such as that which is associated with obesity. In such inflammation, the processes involved are identical to tissue inflammation, but systemic inflammation is not confined to a particular tissue but involves the endothelium and other organ systems. Systemic inflammation may be chronic, and is widely observed in obesity, where many elevated markers of inflammation are observed, including but not limited to: IL-6 (interleukin-6), IL-8 (interleukin-8), IL-18 (interleukin-18), TNF-α (tumor necrosis factor-alpha), CRP (C-reactive protein), insulin, blood glucose, and leptin. Conditions or diseases associated with elevated levels of these markers may be prevented or treated as described herein. In some embodiments, the inflammation may be classified as “low-grade chronic inflammation” in which a two- to threefold increase in the systemic concentrations of cytokines such as TNF-α, IL-6, and CRP is observed. Waist circumference also correlates significantly with systemic inflammatory responses; a predominant factor in this correlation is due to the autoimmune response triggered by adiposity, whereby immune cells “mistake” fatty deposits for infectious agents such as bacteria and fungi. Systemic inflammation may also be triggered by overeating. Meals high in saturated fat, as well as meals high in calories have been associated with increases in inflammatory markers and the response may become chronic if the overeating is chronic.

Inflammation associated disorders that can be treated include NAFLD, NAFL and NASH. NAFLD stands for “non-alcoholic fatty liver disease”, NAFL stands for nonalcoholic fatty liver and NASH stands for nonalcoholic steatohepatitis. Nonalcoholic fatty liver (NAFL) and nonalcoholic steatohepatitis (NASH) are types of NAFLD.

NAFLD is a metabolic dysfunction that stems from insulin resistance-induced hepatic lipogenesis. This lipogenesis increases oxidative stress and hepatic inflammation and is often potentiated by genetic and gut microbiome dysfunction. Risk factors for NAFLD include obesity, gastric bypass surgery, high cholesterol, and type 2 diabetes. Most people have no symptoms but in rare cases, people may experience fatigue, pain, or weight loss. Over time, inflammation and scarring of the liver (cirrhosis) can occur. Liver function tests (blood tests for enzyme levels of increased levels of the liver enzymes such as alkaline phosphatase (ALK), alanine aminotransferase (ALT) and aspartate aminotransferase (AST)), imaging tests (e.g., magnetic resonance imaging (MRI) to identify the anatomical location of damage, MR spectroscopy (MRS) to compare the chemical composition of tissue, ultrasound, CT scanning and isotope examination), and sometimes liver biopsies, are used to diagnose NAFLD, and to tell the difference between NAFL and NASH. Subjects with NAFL have fat in the liver but do not have symptoms of disease, e.g., liver enzymes are not elevated. Subjects with NASH, have inflammation and liver damage, along with fat in the liver, and liver enzymes are generally elevated.

In some aspect, the disease/condition that is treated is metabolic syndrome. Metabolic syndrome is a group of conditions that together raise the risk of coronary heart disease, diabetes, stroke, and other serious health problems. Metabolic syndrome is also called insulin resistance syndrome. Subjects having three or more of the following conditions are susceptible to metabolic syndrome and can benefit by being treated with a compound described herein, especially CA3S: i) a large waistline: this is also called abdominal obesity. Extra fat in the stomach area is a bigger risk factor for heart disease than extra fat in other parts of your body; ii) high blood pressure: if blood pressure rises and stays high for a long time, it can damage the heart and blood vessels. High blood pressure can also cause plaque, a waxy substance, to build up in arteries. Plaque can cause heart and blood vessel diseases such as heart attack or stroke; iii) high blood sugar levels can damage blood vessels and raise the risk of blood clots. Blood clots can cause heart and blood vessel diseases; iv) high blood triglycerides: triglycerides are a type of fat (lipid) found in blood. High levels of triglycerides can raise levels of LDL cholesterol, sometimes called bad cholesterol, raising the risk of heart disease; and v) low HDL cholesterol, sometimes called good cholesterol: blood cholesterol levels are important for heart health. “Good” HDL cholesterol can help remove “bad” LDL cholesterol from blood vessels. “Bad” LDL cholesterol can cause plaque buildup in blood vessels. Each of these symptoms can be treated and brought under control in a subject in need thereof by administering CA of S2CA, preferably S2CA, to the subject, possibly averting full-blown metabolic syndrome, heart disease, stroke, etc.

### Cancer

The present disclosure provides methods for treating at least one of cancer and/or non-cancerous cell transformation by the administration of CA. Examples of such disorders include but are not limited to: Hodgkin's lymphoma, soft tissue sarcoma, leiomyosarcoma, nasopharyngeal carcinoma, Burkitt's lymphoma, T-cell lymphoma, gastric carcinoma, breast cancer e.g., invasive breast cancer), and hierarchically organized carcinoma. Hierarchically organized carcinomas include, but are not limited to, pancreatic ductal adenocarcinoma, urothelial cancer, colorectal cancer, head and neck cancer, non-small cell lung cancer, esophagus cancer, breast cancer, thyroid cancer, oral cancer, cervical cancer, ovarian cancer, and liver cancer (e.g., hepatocellular carcinoma).

Diseases Related to Apoptosis and/or Necrosis

The present disclosure provides a variety of uses for CA, including methods of preventing and/or treating ischemia (e.g., from surgery), necrosis, apoptosis, organ dysfunction, and/or organ failure. The methods include administering to a patient harboring an organ to be treated with an amount of CA that is effective or sufficient to prevent and/or treat dysfunction and/or failure of the organ.

In some aspects, the ischemia that is prevented or treated comprises at least one member selected from cardiac ischemia, brain ischemia, bowel ischemia, limb ischemia, and cutaneous ischemia. In other aspects, the prophylactically treating or treating ischemia comprises reducing one or more of inflammation, tissue necrosis, organ necrosis, risk of stroke, and reperfusion injury in the subject. In additional aspects, the surgery comprises at least one of cardiovascular surgery, heart surgery, and aneurysm surgery.

Aspects of the disclosure also provide methods of preventing or treating dysfunction or failure of one or more organs or organ systems in a subject. The dysfunction or failure may be acute, occurring in a time period of days or weeks (e.g., within 26 weeks, within 13 weeks, within 10 weeks, within 5 weeks, within 4 weeks, within 3 weeks, within 2 weeks, within 1 week, within 5 days, within 4 days, within 3 days, or within 2 days), usually in a person who has no pre-existing disease. Other types of organ dysfunction or failure may occur more slowly (e.g., over a period of months or years) due to a chronic condition.

In some aspects, the one or more organs comprises at least one member selected from the liver, the kidney, the heart, the brain, and the pancreas. In yet other aspects, the dysfunction or failure is Multiple Organ Dysfunction Syndrome (MODS).

### Other Uses

In one aspect, the invention provides the use the compound CA and/or CA3S and/or pharmaceutically acceptable salts thereof, as a medicament.

In some aspects, the invention provides the use of CA or CA3S for the manufacture of a medicament for: reducing lipids in a subject in need thereof; reducing cholesterol and lipid biosynthesis in a subject in need thereof; reducing inflammation in a subject in need thereof; treating diabetes in a subject in need thereof; treating hyperlipidemia in a subject in need thereof; treating atherosclerosis in a subject in need thereof; treating fatty liver disease in a subject in need thereof; treating inflammatory disease in a subject in need thereof; treating cancer in a subject in need thereof; or for any other purpose.

### Sources of the Compounds

CA is readily commercially available.

However, CA can also be synthesized according to the following scheme:

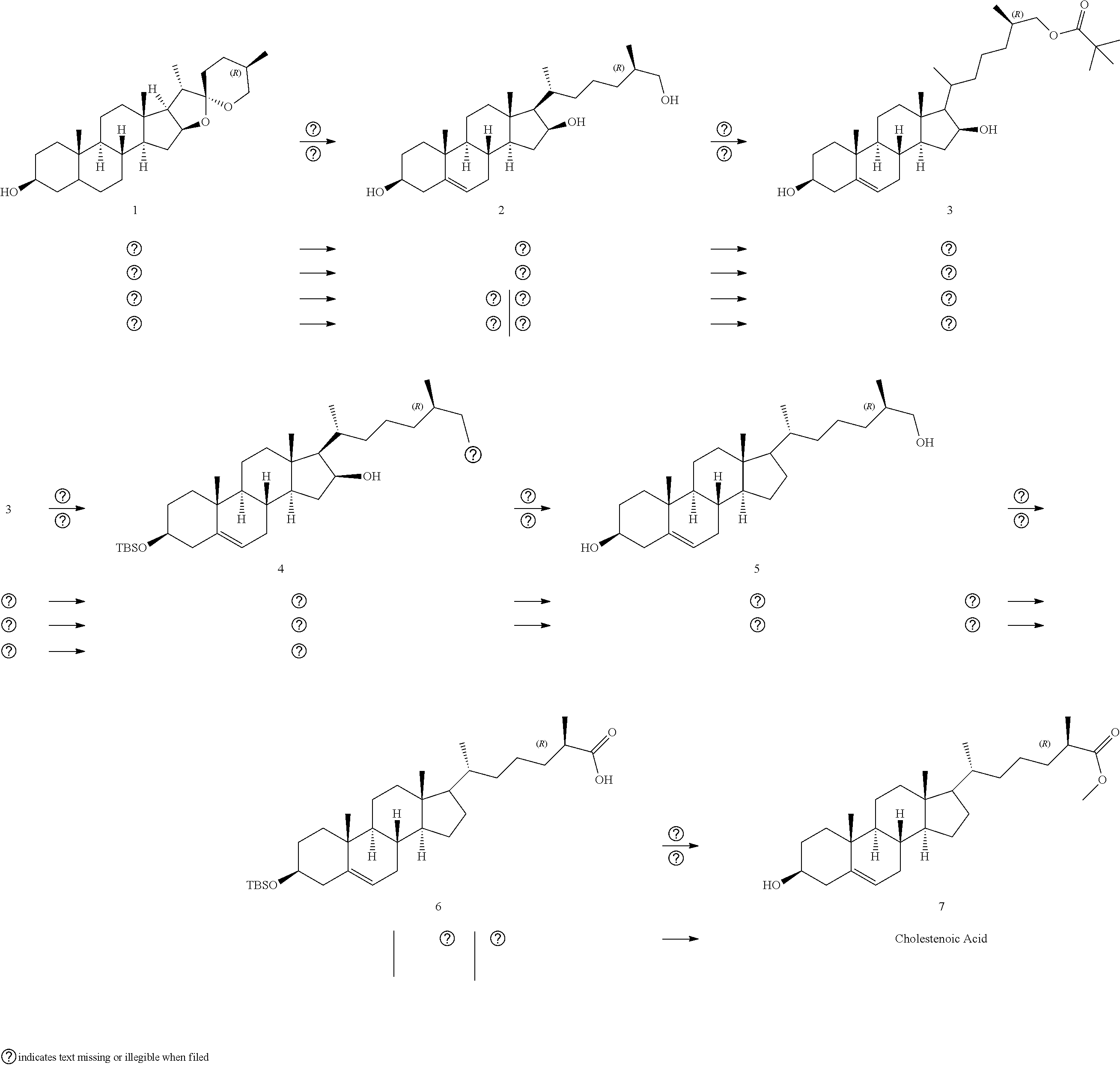

While it is possible to isolate and purify CA3S from living cells, those of skill in the art will recognize that this compound can also be synthesized, e.g., by synthetic chemical means starting with e.g., CA; or by methods which involve the use of recombinant DNA technology (e.g., by using cloned enzymes to carry out suitable modifications of cholesterol or CA or other starting material).

An exemplary chemical synthesis of 3β-hydroxy-5-cholestenoic acid 3-sulfate (CA3S) is provided in the Examples section. Briefly, CA is sulfated using triethylamine-sulfur trioxide (or another suitable sulfonating agent) and the product, CA3S, is purified using column chromatography.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1

In this Example, evidence is provided that CA regulates DNMT1 and suppresses DNMT3a/3b, subsequently decreasing expression of 3-hydroxy-3-methylglutaryl-CoA reductase (HMGR), proprotein convertase subtilisin/kexin type 9 (PCSK9), and fatty acid synthase (FAS). The results suggest that CA may play an important role in the maintenance of intracellular cholesterol and lipid homeostasis in hepatocytes and that CA can be used to lower cholesterol when administered to a subject.

### 2. Materials and Methods

### 2.1. Materials

Cell culture reagents and supplies were purchased from GIBCO™ BRL (Grand Island, NY). HepG-2 cells were obtained from American Type Culture Collection (Rockville, MD). The reagents for quantitative reverse transcription PCR (RT-qPCR) were from AB Applied Biosystems™ (Warrington, UK). The chemicals used in this study were obtained from Sigma Chemical Co. (St. Louis, MO) or Bio-Rad Laboratories (Hercules, CA). All solvents were obtained from Fisher (Fair Lawn, NJ) unless otherwise indicated.

### 2.2. Cell Culture

HepG-2 cells were cultured in DMEM medium supplemented with 10% heat-inactivated fetal bovine serum (FBS), high glucose (HG, 4.5 g/L) at 37° C. in a humidified atmosphere of 5% CO₂.

2.3. Extraction and Determination of DNA and mRNA Levels

After culturing HepG-2 cells in DMEM medium with HG for 72 hours followed by treating with 20 μM CA for 0, 3, 6, 12, and 24 hours, genomic DNA from 5×10⁷ cells were extracted using QIAamp® DNA Mini Kit (QIAGEN, Hilden, Germany). Each sample, 6 μg, was sent to CD Genomics Co., Ltd (New York, USA) for analysis of WGBS. Total RNA was isolated using the Promega SV total RNA isolation system (Madison, WI, USA) with DNase treatment. Each sample, 2 μg, were sent to CD Genomics Co., Ltd (New York, USA) for analysis of RNA sequencing. The same samples, 2 μg, was used for the first-strand cDNA synthesis as recommended by the manufacturer (Invitrogen, Carlsbad, CA). RT-qPCR was performed using SYBR Green as the indicator on ABI 7500 Fast Real-Time PCR System (Applied Biosystems, Foster City, CA). Amplifications of j-actin or GAPDH were used as internal controls. Relative messenger RNA (mRNA) expression was quantified with the comparative cycle threshold (Ct) method using a sutiable primer set and was expressed as 2-ΔΔCt as described previously.

### 2.4. Enzyme Kinetic Study of CA

The enzyme kinetic studies were carried out by Reaction Biology Company. For the DNMT1 activity assay, the substrate solution, 0.001 mg/ml Poly(dI-dC): Poly(dI-dC) in 50 mM Tris-HCl, pH 7.5, 50 mM NaCl, 5 mM EDTA, 5 mM DTT, 1 mM PMSF, 5% glycerol, 0.01% Brij™35, 1% DMSO was used. For the DNMT3a/3b activity assay, 0.0075 mg/ml Lambda DNA in 50 mM Tris-HCl, pH 7.5, 50 mM NaCl, 5 mM EDTA, 5 mM DTT, 1 mM PMSF, 5% glycerol, 1% DMSO, was used. The indicated DNMT1, DNMT3a and DNMT3b were added to the appropriate substrate solution and gently mixed. Amounts of CA ranging from 5.08E-09 to 0.0001 M in DMSO were added to the reaction mixture by using Acoustic Technology (Echo® 550, LabCyte Inc. Sunnyvale, CA). The mixtures were first incubated for 15 min, then ³H-SAM was added to the reaction mixture to initiate the reaction, and the mixture was incubated for 60 min at 30° C. Following incubation, the reaction mixture was finally transferred to filter-paper for detection of radioactivity counts.

### 2.5. Total Cellular and Nuclear Lipid Extraction

HepG-2 cells, 5,000 cells/cm², were cultured in DMEM (HG, 10% FBS) medium for 3 days, and then treated with 20 μM of CA for 0, 3, 6, 12, and 24 hours. After treatment, the cells were washed twice with cold PBS then harvested with 1 ml PBS. For total cell extraction, the cells were collected by centrifuge at 1,000 rpm for 5 min. For nuclei extraction, the nuclear fractions were extracted according to the manufacture's instructions for NE-PER™ Nuclear and Cytoplasmic Extraction Reagents (Thermo Scientific, Rockford, IL). Total proteins in the cell and nuclear fractions were digested with proteinase K (2 mg/ml), incubated at 50° C. with shaking for 3 hours. The total lipids in the total cell and nuclear fractions were extracted with 10 volumes of chloroform:methanol 1:1, vortexed, and sonicated for 30 mins. The extracts were centrifuged at 1,000 rpm for 5 min, the supernatants were dried up by stream nitrogen, then dissolved with 200 μl of methanol, 2 μl of the extracts were used for the CA analysis by LC-MS/MS system as below.

### 2.6. LC-MS/MS Determination of CA Levels in Total Cellular and Nuclear Extracts

Shimadzu LCMS-8600 CL LC-MS system equipped with electrospray ionization (ESI) were used to determine the CA levels. Data were collected and processed using Shimadzu Lab solutions software. Hypersil Gold™ C18 (50 mm×2.1 mm inner diameter, particle size 2.6 μm, Thermo Scientific) was maintained at 40° C. during the analysis. Mobile phase A: 0.1% acetic acid in water and Mobile phase B: methanol:acetonitrile 1:1 was used for gradient elution as follows: 0-8 min, 10% A (linear); 8.01-10 min, 85% A (hold); 10.01-12 min, 10% A (hold). The flow rate was 250 μl/min throughout. The negative ESI parameters were set as follows: The nebulizer gas flow, 2.0 l/min; heating gas flow, 10 l/min; drying gas flow, 10 l/min; interface temperature, 300° C.; interface voltage, 3000 V; desolvation line temperature, 526° C.; heat block temperature, 400° C.; collision gas (argon) pressure, 190 kPa.

### 2.7 Animal Studies

The animal studies were approved by the Institutional Animal Care and Use Committee of McGuire Veterans Affairs Medical Center and conducted in accordance with the Declaration of Helsinki, the Guide for the Care and Use of Laboratory Animals, and all applicable regulations. In this study, a western diet-induced nonalcoholic fatty liver disease (NAFLD) mice model was used. To create the model, eight-week-old C57BL/6J female mice were purchased from the Jackson Laboratory and fed a western diet (TD.88137, Envigo) along with high glucose/fructose water (WDSW) containing 23.1 g/L fructose and 18.9 g/L glucose for 12 weeks. After establishing the model, the mice were separated into three groups based on their weight. For the control mice in each group, they received intravenous injection (IV) with vehicle (DMSO). In contrast, the treatment group mice were intravenously injected with 10 mg/kg of CA (dissolved in DMSO) with a total volume of less than 100 μl. During the treatment period, injections were administered every two days. All mice were housed under identical conditions in an aseptic facility with a 12-hour light/12-hour dark cycle and provided with free access to water and food (WDSW). Before scarification, the mice fasted overnight. Blood samples were collected, and the serum enzymatic activities of alkaline phosphatase (ALK), alanine aminotransferase (ALT), and aspartate aminotransferase (AST) were measured in the clinical laboratory at McGuire Veterans Affairs Medical Center.

### 2.7. Analysis of Whole Genome Bisulfite Sequencing (WGBS)

Each sample, 1 μg of genomic DNA was fragmented by sonication to a mean size of approximately 200-400 bp, and subsequently used for WGBS library construction using Acegen Bisulfite-Seq Library Prep Kit (Acegen) following the manufacturer's instructions. The methylated adapter-ligated DNAs were purified using 0.8×Agencourt AMPure™ XP magnetic beads and subjected to bisulfite conversion by ZYMO EZ DNA Methylation-Gold™ Kit (Zymo Research Corporation, CA, USA). The converted DNAs were then amplified using 25 μl KAPA HiFi™ HotStart Uracil+ ReadyMix (2×) and 8-bp index primers with a final concentration of 1 μM each. The constructed WGBS libraries were then analyzed by Agilent 2100 Bioanalyzer and quantified by a Qubit fluorometer with Quant-iT™ dsDNA HS Assay Kit (Invitrogen), and finally sequenced on Illumina® Hiseq X™ Ten sequencer. After the preparation of the library, Qubit 2.0 and Agilent 2100 were used respectively to detect the concentration of the library and the Insert Size, and the effective concentration (>2 nM) of the library was quantitatively determined by Q-PCR to ensure the library quality.

Samples were sequenced using the Illumina® HiSeq sequencing platform. Raw data generated on the sequencing platform contained a small percentage of low-quality data, which was then filtered to get high-quality data. Bsmap software was used to perform alignments of bisulfite-treated reads to a reference genome (GRCh37). Metilene software was used to identify differentially methylated regions (DMRs). DAVID software (website located at david.ncifcrf.gov/) was used to test the statistical enrichment of DMR related genes in the Gene Ontology (GO) and Kyoto Encyclopedia of Genes and Genomes (KEGG) pathways.

### 2.8 Transcriptional Profiling and Data Analysis

Total RNA was extracted and purified from HepG-2 cells using SV total RNA isolation system (Promega, Madison, WI). Messenger RNA was purified from total RNA using poly-T oligo-attached magnetic beads. After fragmentation, the first strand cDNA was synthesized using random hexamer primers, followed by the second strand cDNA synthesis using either dUTP for directional library or dTTP for non-directional library. The library was checked with Qubit and real-time PCR for quantification and bioanalyzer for size distribution detection. Quantified libraries will be pooled and sequenced on Illumina® platforms, according to effective library concentration and data amount. Raw data (raw reads) of fastq format were first processed through in-house perl scripts. In this step, clean data (clean reads) were obtained by removing reads containing adapter, reads containing ploy-N and low-quality reads from raw data. At the same time, Q20, Q30 and GC content of the clean data were calculated. All the downstream analyses were based on clean data with high quality. The clean data were aligned to reference genome (GRCh37) using Hisat2 v2.0.5 software. Differential expression genes (DEGs) were performed using the DESeq2 R package (1.20.0). DAVID software (website located at david.ncifcrf.gov/) was used to test the statistical enrichment of DMR related genes in the GO and KEGG pathways.

### 2.9. Western Blot Analysis

Specific proteins were analyzed by western blot. The proteins were extracted using M-PER™ Mammalian Protein Extraction Reagents (Fisher Scientific). For each sample, 20 μg of proteins were separated on 8%-12% SDS polyacrylamide gel electrophoresis (SDS-PAGE) gels. Electrophoresis was performed at 100V for 15 min and 200V for another 25 min in a Bio-Rad mini-gel system. After electrophoresis, samples were transferred onto a polyvinylidene difluoride (PVDF) membrane at 30V for 50 min. The specific proteins on the membrane were detected by incubation with anti-AMPKα1/2 (Santa Cruz Biotechnologies) primary antibody at 4° C. overnight, followed by further incubation with an appropriate horseradish peroxidase (HRP)-conjugated secondary antibody (Bio-Rad) at room temperature for 1 hr. Each positive band was quantified by Advanced Image Data Analyzer (Aida, Straubenhardt, Germany).

### 2.9. Untargeted Lipidomics Analysis

HepG-2 cells were cultured in DMEM medium with HG for 72 hours followed by treating with 20 μM CA for 24, 48, and 72 hours. The cells were harvested with 500 μl 1× PBS, and sent to Creative Proteomics Co., Ltd (New York, USA) for untargeted lipidomics analysis. Samples were thawed and 1.5 mL chloroform:methanol (2:1, v/v) added to sample, vortexed for 1 min, and followed by sonication for 30 min, 4° C. Then centrifuge 10 min at 12,000 rpm, 4° C., transfer the lower phase to a new tube, dry under the nitrogen. The dried extract with 200 μl of isopropyl alcohol:methanol (1:1, v/v); add 5 μl LPC (12:0) for internal standard. Finally, centrifuge 10 min at 12,000 rpm, 4° C.; transfer the supernatant for LC-MS analysis. Separation is performed by UltiMate™ 3000 LC combined with Q Exactive™ MS (Thermo) and screened with ESI-MS. The LC system is comprised of ACQUITY UPLC® BEH C18 (100×2.1 mm×1.7 μm) with UltiMate™ 3000 LC. The mobile phase is composed of solvent A (60% ACN+40% H₂O+10 mM HCOONH₄) and solvent B (10% ACN+90% isopropyl alcohol+10 mM HCOONH₄) with a gradient elution (0-10.5 min, 30%-100% B; 10.5 min-12.5 min, 100% B; 12.5-12.51 min, 100%-30% B; 12.51-16.0 min, 30% B). The flow rate of the mobile phase is 0.3-1 mL·min. The column temperature is maintained at 40° C., and the sample manager temperature is set at 4° C. Mass spectrometry parameters in ESI+ and ESI− mode are listed as follows: ESI+: Heater Temp 300° C.; Sheath Gas Flow rate, 45 arb; Aux Gas Flow Rate, 15 arb; Sweep Gas Flow Rate, larb; spray voltage, 3.0 KV; Capillary Temp, 350° C.; S-Lens RF Level, 30%. ESI−: Heater Temp 300° C., Sheath Gas Flow rate, 45 arb; Aux Gas Flow Rate, 15 arb; Sweep Gas Flow Rate, larb; spray voltage, 3.2 KV; Capillary Temp, 350° C.; S-Lens RF Level, 60%.

### 2.10. Statistics

Data are reported as the mean±standard deviation. Where indicated, data were subjected to t-test analysis and determined to be significantly different if p<0.05.

### 3. Results

### 3.1. CA as a Ligand of DNMTs in Hepatocyte Nuclei

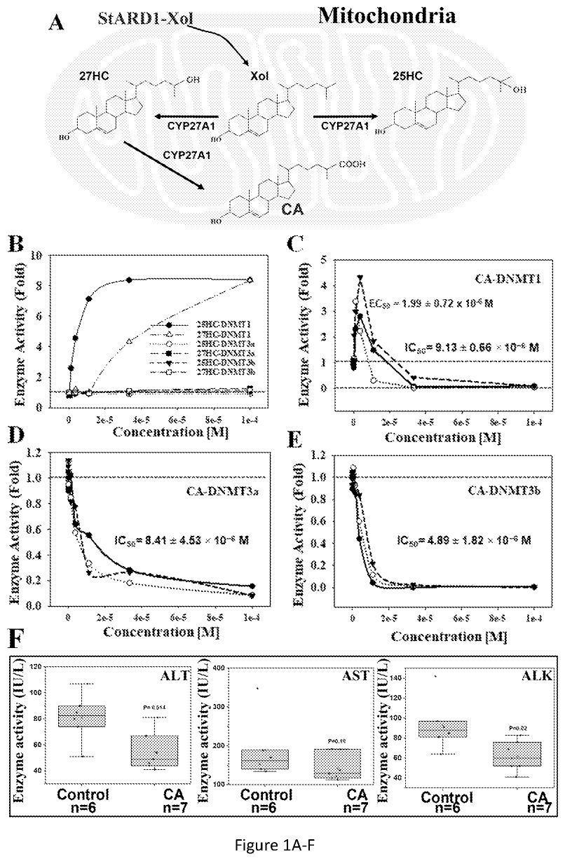

27HC, 25HC, and CA are biosynthesized in mitochondria have been previously reported as naturally occurring ligands for LXRs (FIG. 1A). 27HC and 25HC play important roles in lipid metabolism, inflammatory responses, and cell apoptosis. Our previous report showed that 25HC and 27HC both activate DNMT1 activity, regulate lipid metabolism and cell apoptosis through DNA methylation. Enzyme kinetic effects of 25HC and 27HC on DNMTs activity are summarized in FIG. 1B. To determine if CA affects DNA methyltransferase activity, recombinant DNMT1, DNMT3a, and DNMT3b were used for enzyme kinetic studies. The results demonstrated that CA specifically activates DNMT1 at low concentration with EC₅₀=1.99±0.72×10⁻⁶ M and inhibits activity at higher concentration with IC₅₀=9.13±0.66×10⁻⁶ M (FIG. 1C), and specifically inhibits DNMT3a, and DNMT3b activities with IC₅₀=8.41±4.53×10⁻⁶ M and IC₅₀=4.89±1.82×10⁻⁶ M, respectively, as shown in FIGS. 1D and 1E. As a positive control, S-adenosyl homocysteine inhibited DNMT1 activity by 95% at 1 μM, as previously reported. The results demonstrated that CA is a potent inhibitor of DNMT3a/3b and DNMT1 at high concentration but is an activator of DNMT1 at low concentration. DNMT1 and DNMT3b are co-localized in nuclei.

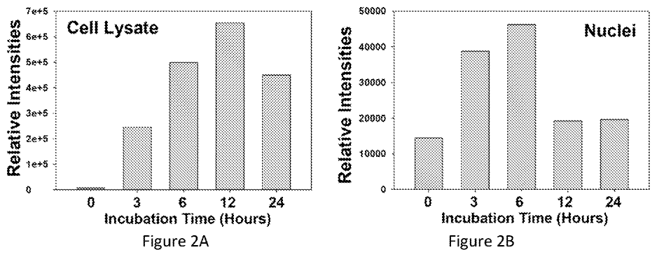

To further confirm the physiological significance, the intracellular distribution following the addition of CA to culture medium was investigated using LC-MS/MS analysis. Before the exogenous addition of CA, the intracellular levels were ˜0.5 ng/million cells; following the addition of 20 μM CA, the levels were increased by 85-fold at 12 hours (FIGS. 2A and B). Interestingly, CA maximally accumulated in the cellular nuclei at 3 and 6 hours post addition, increased by 4-fold at 6 hours (FIGS. 2A and B). The results indicate that nuclei may be the sub-organelle target for CA. To assess the in vivo toxicity of CA, a dosage of 10 mg/kg CA was administered via injection using the NAFLD mouse model. The results revealed that even after two weeks of treatment, the mice remained in good health, indicating no adverse effects of CA on their well-being. Additionally, the serum activities of ALT and ALK exhibited a significant decrease, reaching levels within the normal range (FIG. 1F). This data suggests that CA treatment was not only well-tolerated but also benefited NAFLD recovery.

### 3.2. Effects of CA on the Whole Genome-Wide DNA Methylation in Human Hepatocytes

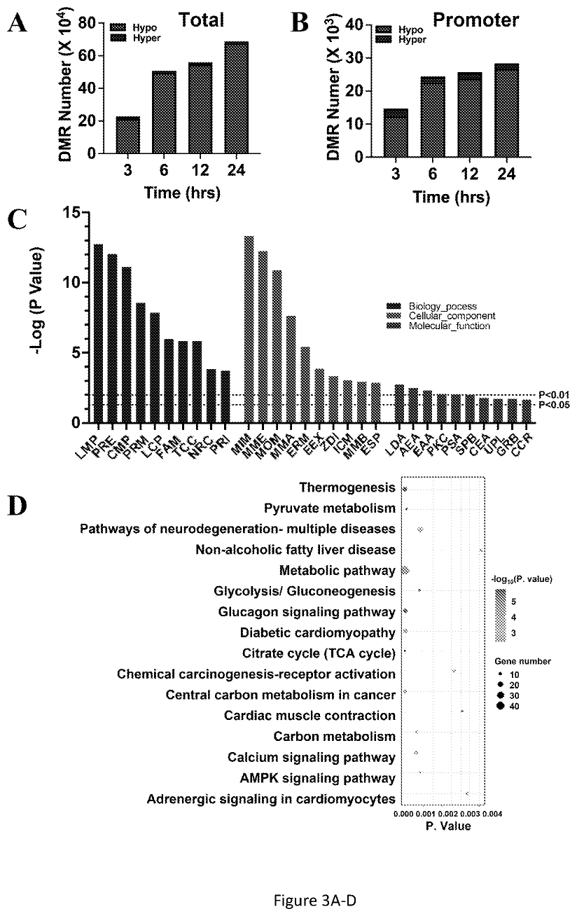

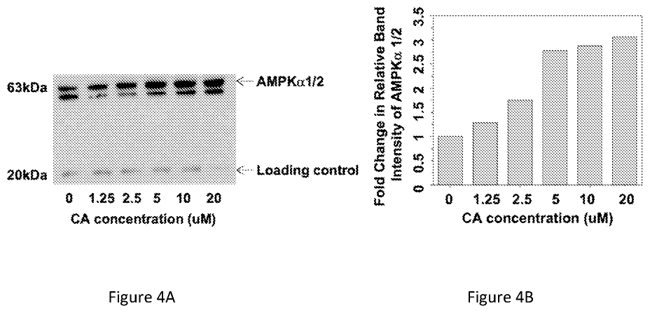

To determine the possible cellular functions of 5 mCpG demethylation, HepG-2 cells were treated with 20 μM CA for 0, 3, 6, 12, and 24 hours and harvested for the construction of bisulfite-treated genomic DNA libraries. In these libraries, more than 88% bases have scores >Q30 for single and paired end reads. The depth and density of sequencing were enough for a high-quality genome-wide methylation analysis. Moreover, the efficiencies of bisulfite conversion, represented by lambda DNA to the libraries, were over 99%, providing reliable and accurate results for the WGBS (data not shown). CpG methylation and demethylation are well documented to relate with gene expression. A total of 227,565 DMRs were detected at 3 hours following CA treatment (11,818 were hyper-methylated, and 215,747 were hypo-methylated) from the libraries from the cells that were treated with 20 μM CA compared to vehicle control. Moreover, at 6 hours 508,222 (10,537 were hyper-methylated, and 497,685 were hypo-methylated), at 12 hours 559,574 (14,658 were hyper-methylated, and 544,916 were hypo-methylated), and at 24 hours 687,859 (8,808 were hyper-methylated, and 679,051 were hypo-methylated) (FIG. 3A). Further analysis of DMRs showed that of 14,754 DMRs (2,323 were hyper-methylated, and 12,431 were hypo-methylated) were in promoter region at 3 hours, 24,370 (1,749 were hyper-methylated, and 22,621 were hypo-methylated) at 6 hours, 25,704 (1,891 were hyper-methylated, and 23,813 were hypo-methylated) at 12 hours, 28,356 (1,594 were hyper-methylated, and 26,762 were hypo-methylated) at 24 hours (FIG. 3B). In order to analyze the function of hypo-methylated DMRs in promoter regions, 24,370 DMRs that were treated with CA for 6 hours were enriched into GO and KEGG database. The results showed that the major GO processes are lipid metabolism related, including lipid metabolic process (LMP), positive regulation of ERK1 and ERK2 cascade (PRE), carbohydrate metabolic process (CMP), positive regulation of MAPK cascade (PRM), lipid catabolic process (LCP), fatty acid metabolic process (FAM) (FIG. 3C). For KEGG database, these DMRs were significantly (p<0.05) enriched into metabolic signaling pathways, including calcium, AMPK, NAFLD, chemical carcinogenesis-receptor activation, and glucagon signaling pathways (FIG. 3D). Among these pathways, calcium and AMPK signaling are hypothesized to be master pathways regulating cell survival, antioxidants, anti-apoptosis, energy metabolism, and lipid homeostasis. CA increased demethylation of 5 mCpG in promoter regions of 13 genes involved in calcium signaling pathway (Table 1), 9 genes involved in NAFLD pathway (Table 2), 9 genes involved in AMPK signaling pathway (Table 3),12 genes involved in glucagon signaling pathway (Table 4), and 9 genes involved in chemical carcinogenesis receptor activation pathway (Table 5).The chromosome and sequence location of the hypomethylated CpG by CA in promoter regions are compared in Tables 1-5. The details of KEGG pathways that were enriched by hypo-methylated promoter regions at 6 hours post treatment are listed in Table 6. The results indicate that the global regulatory mechanisms of CA are through demethylation of 5 mCpG in promoter regions of the key genes involved in calcium channels and calmodulin families. In order to confirm the effect of CA on the protein levels of AMPK, HepG-2 cells were treated with 20 μM CA in high glucose media for 24 hours and total protein was extracted from the treated cells. The extracted proteins, 20 μg each sample, were used for western blot analysis. The results demonstrated that CA significantly increased the protein levels of AMPK in a concentration-dependent manner as shown in FIGS. 4A and B.

TABLE 1
Demethylation of 5mCpG in promoter regions of
calcium signaling pathway (P = 0.0026 at 6 h)

| DMR Location in Promoter Region | | | | DMR (Methylation %) | | | |
|---|---|---|---|---|---|---|---|
| Gene Name | Chromosome | Start | End | 3 h | 6 h | 12 h | 24 h |
| ADRA1 | chr8 | 26,722,718 | 26,723,338 | −30.28 | −41.25 | −30.57 | −30.81 |
| ADRA1B | chr5 | 159,343,856 | 159,343,903 | 0 | −33.89 | 0 | 0 |
| CACNA1D | chr3 | 53,529,276 | 53,529,335 | −55.12 | −69.06 | 0 | 0 |
| CALML3 | chr10 | 5,566,643 | 5,566,944 | −27.06 | −47.29 | −30.32 | −26.91 |
| CALML4 | chr15 | 68,498,384 | 68,498,808 | −21.08 | −31.79 | −21.6 | −21 |
| FGF1 | chr5 | 142,025,495 | 142,025,537 | −54.6 | −37.79 | −48.58 | 0 |
| FGF8 | chr10 | 103,539,541 | 103,539,685 | −19.97 | −53.29 | −37.87 | 0 |
| FGFR2 | chr10 | 123,292,667 | 123,292,700 | −26.28 | −47.13 | 0 | −24.29 |
| NTRK3 | chr15 | 88,749,804 | 88,749,813 | 0 | −49.89 | −53.95 | −53.82 |
| PHKG1 | chr7 | 56,161,011 | 56,161,115 | −15.73 | −41.81 | −24.54 | −33.52 |
| SPHK2 | chr19 | 49,127,579 | 49,128,650 | 0 | −60.87 | −18.02 | −45.29 |
| VDAC2 | chr10 | 76,968,663 | 76,968,696 | −27.41 | −40.06 | −19.71 | −26.13 |

TABLE 2
Demethylation of 5mCpG in promoter regions of
AMPK signaling pathway (P = 0.0008 at 6 h)

| DMR Location in Promoter Region | | | | DMR (Methylation %) | | | |
|---|---|---|---|---|---|---|---|
| Gene Name | Chromosome | Start | End | 3 h | 6 h | 12 h | 24 h |
| ADRA1A | chr8 | 26,722,718 | 26,723,338 | −30.28 | −41.25 | −30.57 | −30.81 |
| CAMKK2 | chr12 | 121,713,910 | 121,713,989 | 0 | −37.97 | −23.88 | 0 |
| CPT1A | chr11 | 68,610,327 | 68,610,381 | −17.85 | −41.69 | −16.93 | −29.33 |
| CPT1C | chr19 | 50,194,334 | 50,194,456 | −18.8 | −34.41 | −24.72 | −24.09 |
| CREB3L4 | chr1 | 153,938,859 | 153,938,897 | 0 | −36.38 | 0 | −35.93 |
| FBP1 | chr9 | 97,401,415 | 97,401,438 | 0 | −39.18 | 0 | −28.62 |
| LEP | chr7 | 127,880,823 | 127,880,836 | 0 | −35.18 | −62.7 | 0 |
| MTOR | chr1 | 11,323,660 | 11,323,854 | −20.34 | −37.3 | −27.13 | 0 |
| PPARG | chr3 | 12,352,022 | 12,393,324 | 0 | −37.32 | −27.41 | −27.57 |

TABLE 3
Demethylation of 5mCpG in promoter regions of
NAFLD signaling pathway (P = 0.0041 at 6 h)

| DMR Location in Promoter Region | | | | DMR (Methylation %) | | | |
|---|---|---|---|---|---|---|---|
| Gene Name | Chromosome | Start | End | 3 h | 6 h | 12 h | 24 h |
| ADRA1A | chr8 | 26,722,718 | 26,723,338 | −30.28 | −41.25 | −30.57 | −30.81 |
| COX7B | chrX | 77,154,454 | 77,154,461 | −30.9 | −83.31 | 0 | 0 |
| COX8A | chr11 | 63,740,862 | 63,741,002 | −25.87 | −35.3 | 0 | −34.15 |
| LEP | chr7 | 127,880,823 | 127,880,836 | 0 | −35.18 | −62.7 | 0 |
| PPARG | chr3 | 12,352,022 | 12,393,324 | 0 | −37.32 | −27.41 | −27.57 |
| SDHA | chr5 | 217,194 | 217,240 | 0 | −37.82 | 0 | −23.5 |
| SDHB | chr1 | 17,381,359 | 17,381,392 | 0 | −39.54 | −38.35 | 0 |
| UQCRC1 | chr3 | 48,647,954 | 48,648,002 | −23.76 | −56.12 | −23.36 | −26.32 |

TABLE 4
Demethylation of 5mCpG in promoter regions of glucagon
signaling pathway (P = 0.000001 at 6 h)

| DMR Location in Promoter Region | | | | DMR (Methylation %) | | | |
|---|---|---|---|---|---|---|---|
| Gene Name | Chromosome | Start | End | 3 h | 6 h | 12 h | 24 h |
| CALML3 | Chr10 | 5,566,643 | 5,566,944 | −27.05 | −47.29 | −30.32 | −26.91 |
| CALML4 | Chr19 | 68,498,384 | 68,498,808 | −21.08 | −31.79 | −21.60 | −21.00 |
| CPT1A | Chr5 | 68,610,327 | 68,610,381 | 17.85 | −41.69 | −16.93 | −29.33 |
| CPT1C | Chr16 | 50,194,334 | 50,194,456 | −18.80 | −34.41 | −24.72 | −24.09 |
| CREB3L4 | Chr5 | 153,938,859 | 153,938,897 | 0 | −36.38 | 0 | −35.93 |
| FBP1 | Chr7 | 97,401,415 | 97,401,438 | 0 | −39.18 | 0 | −28.62 |
| LDHA | Chr21 | 18,415,186 | 18,415,286 | −29.00 | −35.80 | −36.43 | −32.94 |
| LDHAL6A | Chr12 | 18,475,648 | 18,476,908 | 0 | −38.96 | 0 | −22.36 |
| LDHC | Chr7 | 18,433,265 | 18,433,366 | −20.18 | −31.45 | 0 | −31.77 |
| PDHA2 | Chr5 | 96,761,255 | 96,761,265 | 0 | −39.34 | −28.93 | −38.56 |
| PDHB | Chr7 | 58,420,530 | 58,420,624 | −21.86 | −42.31 | 0 | −26.74 |
| PHKG1 | Chr9 | 56,161,011 | 56,161,115 | −15.73 | −41.81 | −24.54 | −33.52 |

TABLE 5
Demethylation of 5mCpG in promoter regions of chemical carcinogenesis-
receptor activation pathway (P = 0.0027 at 6 h)

| DMR Location in Promoter Region | | | | DMR (Methylation %) | | | |
|---|---|---|---|---|---|---|---|
| Gene Name | Chromosome | Start | End | 3 h | 6 h | 12 h | 24 h |
| BCL6 | Chr24 | 187,455,336 | 187,455,954 | −20.42 | −43.30 | −33.25 | −31.52 |
| CACNA1D | Chr7 | 53,529,276 | 53,529,335 | −55.12 | −69.10 | 0 | 0 |
| CREB3L4 | Chr5 | 153,938,859 | 153,938,897 | 0 | −36.38 | 0 | −35.93 |
| CYP1A1 | Chr5 | 75,019,335 | 75,019,387 | −30.07 | −55.38 | 0 | −27.04 |
| ESR2 | Chr14 | 64,751,715 | 64,751,75 | 0 | −33.80 | −28.97 | −24.69 |
| FGF8 | Chr10 | 103,539,541 | 103,539,685 | −19.97 | −53.29 | −37.87 | 0 |
| MIR29A | Chr7 | 130,562,558 | 130,562,743 | −15.46 | −32.98 | −22.09 | −26.93 |
| MIRLET7B | Chr22 | 46,508,476 | 46,508,565 | 0 | −34.85 | 0 | −24.75 |
| MTOR | Chr1 | 11,323,660 | 11,323,854 | −20.34 | −37.30 | −27.13 | 0 |

TABLE 6
Significant Enrichment KEGG Pathways of Hypomethylated
DMGs in Promoter Region under CG Context for 6 Hours

| Pathways | P-value |
|---|---|
| Thermogenesis | 0.00000125 |
| Glucagon signaling pathway | 0.00000136 |
| Citrate cycle (TCA cycle) | 0.00000788 |
| Metabolic pathways | 0.00001754 |
| Central carbon metabolism in cancer | 0.00001823 |
| Diabetic cardiomyopathy | 0.00002907 |
| Pyruvate metabolism | 0.00011314 |
| Calcium signaling pathway | 0.00060195 |
| Carbon metabolism | 0.00061242 |
| Glycolysis/Gluconeogenesis | 0.00080242 |
| AMPK signaling pathway | 0.00081197 |
| Pathways of neurodegeneration - multiple diseases | 0.00081544 |
| Chemical carcinogenesis - receptor activation | 0.00265260 |
| Cardiac muscle contraction | 0.00310245 |
| Adrenergic signaling in cardiomyocytes | 0.00336053 |
| Non-alcoholic fatty liver disease | 0.00410393 |
| Parkinson disease | 0.00452329 |
| Prion disease | 0.00548828 |
| Adipocytokine signaling pathway | 0.00555534 |
| Oxidative phosphorylation | 0.00670030 |
| Cysteine and methionine metabolism | 0.00983538 |
| Alzheimer disease | 0.01090531 |
| Chemical carcinogenesis - reactive oxygen species | 0.01156387 |
| Huntington disease | 0.01242595 |
| Salivary secretion | 0.01800666 |
| Glycerophospholipid metabolism | 0.02300423 |
| Insulin signaling pathway | 0.02593418 |
| Apelin signaling pathway | 0.02759681 |
| Mitophagy - animal | 0.03109435 |
| HIF-1 signaling pathway | 0.03432974 |
| Cholesterol metabolism | 0.04946439 |

### 3.3. Effects of CA on the Gene Expression at Transcriptional Level in Human Hepatocytes

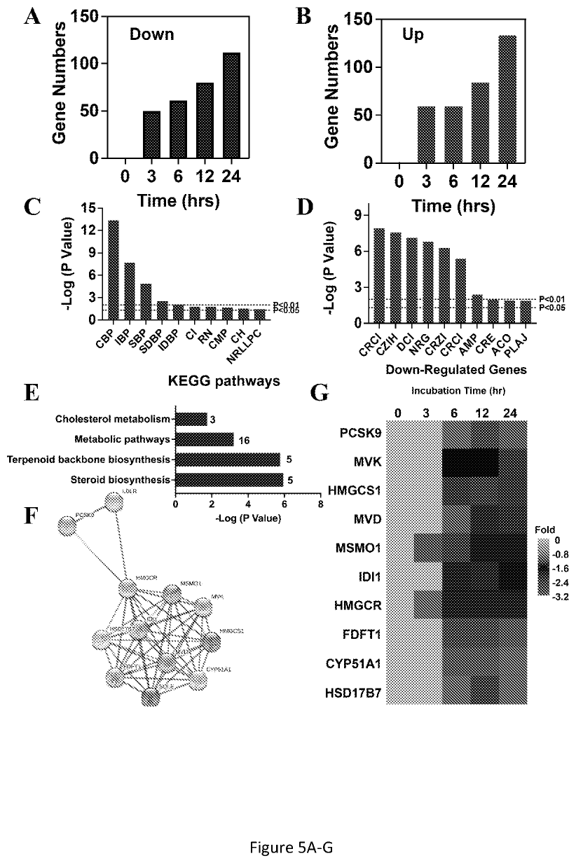

DNA methylation or demethylation is one of the important mechanisms for regulating gene expression. To examine the effect of CA on whole gene expression in human hepatocytes, mRNA sequencing was used. The results showed that treatment of HepG-2 cells with CA significantly modulated numerous gene clusters. There were 109 different genes (DEGs) regulated by CA (59 were up-regulated, 50 were down-regulated) at 3 hours post treatment, 120 DEGs (59 were up-regulated, 61 were down-regulated) at 6 hours, 164 DEGs (84 were up-regulated, 80 were down-regulated) at 12 hours, 245 DEGs (133 were up-regulated, 112 were down-regulated) at 24 hours (FIGS. 5A and B). The up-regulated genes by CA at 6 hours are shown in Table 7 and those down-regulated genes are shown in Table 8. To analyze the biological functions of these DEGs, the raw data from 6 hours treatment were enriched into the GO and KEGG database. The results showed that 61 down-regulated genes were significantly (P<0.05) enriched in lipids biosynthesis process, including cholesterol biosynthetic process (CBP), sterol biosynthetic process (SBP), steroid biosynthetic process (SDBP), cholesterol import (CI) (FIG. 5C). While the 59 up-regulated genes were enriched into ion process, including cellular response to copper ions (CRCI), cellular zinc ion homeostasis (CZIH), and detoxification of copper ions (DCI) (FIG. 5D). The 61 down-regulated genes were significantly (P<0.05) enriched into 4 KEGG pathways, steroid biosynthesis, terpenoid backbone biosynthesis, metabolic pathways, and cholesterol metabolism (FIG. 5E). The gene networks were constructed by STRING tool (website located at string-db.org/) as shown in FIG. 3F. The top down-regulated genes are list in FIG. 5G. The results indicated that CA significantly down-regulates key genes involved in steroid biosynthesis pathways in hepatocytes.

TABLE 7
Up Regulated Gene List of HpG-2 Cells Treated by CA for 6 hours

| | Fold | |
|---|---|---|
| Gene Symbol | Change | Gene Name or Function |
| GMPR2 | 3.88 | Guanosine monophosphate reductase 2 |
| RPS17 | 3.79 | Ribosomal protein S17 |
| CD99 | 3.60 | CD99 molecule (Xg blood group) |
| ACTN4 | 3.26 | Actinin alpha 4 |
| VARS1 | 3.03 | Valyl-trna synthetase 1 |
| EEF1A1P13 | 2.82 | Eukaryotic translation elongation factor 1 alpha 1 pseudogene |
| | | 13 |
| SLC39A7 | 2.59 | Solute carrier family 39 member 7 |
| MT1F | 2.51 | Metallothionein 1F |
| ARHGAP17 | 2.50 | Rho gtpase activating protein 17 |
| MT1G | 2.43 | Metallothionein 1G |
| PSME2 | 2.33 | Proteasome activator subunit 2 |
| GBA | 2.25 | Glucosylceramidase beta 1 |
| MCRIP1 | 2.17 | MAPK regulated corepressor interacting protein 1 |
| MT1X | 2.15 | Metallothionein 1X |
| FLII | 2.04 | FLII actin remodeling protein |
| CYP1A1 | 1.97 | Cytochrome P450 family 1 subfamily A member 1 |
| VPS11 | 1.96 | VPS11 core subunit of CORVET and HOPS complexes |
| GTF2H1 | 1.85 | General transcription factor IIH subunit 1 |
| UGT2A3 | 1.83 | UDP glucuronosyltransferase family 2 member A3 |
| MT1E | 1.81 | Metallothionein 1E |
| CUTA | 1.76 | Cuta divalent cation tolerance homolog |
| GNL1 | 1.59 | G protein nucleolar 1 (putative) |
| PTGDR2 | 1.58 | Prostaglandin D2 receptor 2 |
| RPS14P1 | 1.50 | Ribosomal protein S14 pseudogene 1 |
| MTMR11 | 1.48 | Myotubularin related protein 11 |
| NDUFS1 | 1.45 | NADH:ubiquinone oxidoreductase core subunit S1 |
| MT2A | 1.44 | Metallothionein 2A |
| DLG5 | 1.44 | Discs large MAGUK scaffold protein 5 |
| PHF1 | 1.42 | PHD finger protein 1 |
| IRF9 | 1.42 | Interferon regulatory factor 9 |
| U2AF1 | 1.40 | U2 small nuclear RNA auxiliary factor 1 |
| PRPF31 | 1.38 | Pre-mrna processing factor 31 |
| GBP1 | 1.37 | Guanylate binding protein 1 |
| NPIPA2 | 1.32 | Nuclear pore complex interacting protein family member A2 |
| ACTA1 | 1.32 | Actin alpha 1, skeletal muscle |
| EEF1B2 | 1.29 | Eukaryotic translation elongation factor 1 beta 2 |
| RNF187 | 1.25 | Ring finger protein 187 |
| FST | 1.25 | Follistatin |
| MINCR | 1.24 | MYC-induced long non-coding RNA |
| HSPA1A | 1.22 | Heat shock protein family A (Hsp70) member 1A |
| LOC112268009 | 1.19 | Loc112268009 |
| SPSB2 | 1.18 | Spla/ryanodine receptor domain and SOCS box containing 2 |
| RCBTB2 | 1.18 | RCC1 and BTB domain containing protein 2 |
| HSPA1B | 1.16 | Heat shock protein family A (Hsp70) member 1B |
| EHMT2 | 1.16 | Euchromatic histone lysine methyltransferase 2 |
| SYNPO | 1.14 | Synaptopodin |
| SBNO2 | 1.13 | Strawberry notch homolog 2 |
| SYBU | 1.09 | Syntabulin |
| STAT5A | 1.09 | Signal transducer and activator of transcription 5A |
| TPM1-AS | 1.08 | TPM1 antisense RNA |
| FZD1 | 1.06 | Frizzled class receptor 1 |
| SERTAD1 | 1.06 | SERTA domain containing 1 |
| NEK10 | 1.06 | NIMA related kinase 10 |
| INPP5K | 1.04 | Inositol polyphosphate-5-phosphatase K |
| SGK1 | 1.03 | Serum/glucocorticoid regulated kinase 1 |
| LFNG | 1.02 | LFNG O-fucosylpeptide 3-beta-N- |
| | | acetylglucosaminyltransferase |
| PALLD | 1.02 | Palladin, cytoskeletal associated protein |
| TJP1 | 1.02 | Tight junction protein 1 |

TABLE 8
Down Regulated Gene List of HpG-2 Cells Treated by CA for 6 hours

| Gene Symbol | Fold Change | Gene Name or Function |
|---|---|---|
| PSMB3 | −4.67 | Proteasome 20S subunit beta 3 |
| CPSF1 | −4.45 | Cleavage and polyadenylation specific factor 1 |
| PAK1IP1 | −3.71 | Proprotein convertase subtilisin/kexin type 9 |
| NOP9 | −2.89 | Leukotriene B4 receptor 2 |
| GFUS | −2.73 | GDP-L-fucose synthase |
| ID3 | −2.65 | Inhibitor of DNA binding 3 |
| MTCH2 | −2.50 | Mitochondrial carrier 2 |
| IBTK | −2.44 | Inhibitor of Bruton tyrosine kinase |
| MSMO1 | −2.30 | Methylsterol monooxygenase 1 |
| HMGCS1 | −2.12 | 3-hydroxy-3-methylglutaryl-coa synthase 1 |
| GTPBP6 | −2.08 | GTP binding protein 6 (putative) |
| PUF60 | −2.01 | Poly(U) binding splicing factor 60 |
| NEU4 | −1.99 | Neuraminidase 4 |
| FBL | −1.87 | Fibrillarin |
| IDI1 | −1.86 | Isopentenyl-diphosphate delta isomerase 1 |
| YWHAE | −1.84 | Tyrosine 3-monooxygenase/tryptophan 5-monooxygenase |
| | | activation protein epsilon |
| ANGPTL8 | −1.77 | Angiopoietin like 8 |
| DCAF11 | −1.74 | DDB1 and CUL4 associated factor 11 |
| SQLE | −1.73 | Squalene epoxidase |
| TBCE | −1.67 | Tubulin folding cofactor E |
| PTEN | −1.64 | Phosphatase and tensin homolog |
| SNHG32 | −1.62 | Small nucleolar RNA host gene 32 |
| TINF2 | −1.58 | TERF1 interacting nuclear factor 2 |
| RN7SK | −1.58 | RNA component of 7SK nuclear ribonucleoprotein |
| MVK | −1.57 | Mevalonate kinase |
| HMGCR | −1.55 | 3-hydroxy-3-methylglutaryl-coa reductase |
| IPO4 | −1.51 | Importin 4 |
| SPC25 | −1.51 | SPC25 component of NDC80 kinetochore complex |
| LDLR | −1.46 | Low density lipoprotein receptor |
| PRSS8 | −1.46 | Serine protease 8 |
| COG5 | −1.44 | Component of oligomeric golgi complex 5 |
| LPCAT1 | −1.43 | Lysophosphatidylcholine acyltransferase 1 |
| PNPLA3 | −1.42 | Patatin like phospholipase domain containing 3 |
| LINC02119 | −1.39 | Long intergenic non-protein coding RNA 2119 |
| RUSC1-AS1 | −1.37 | RUSC1 antisense RNA 1 |
| NUP160 | −1.36 | Nucleoporin 160 |
| ABCF1 | −1.35 | ATP binding cassette subfamily F member 1 |
| LOC107985276 | −1.32 | Zinc finger protein 440-like |
| ATG16L1 | −1.32 | Autophagy related 16 like 1 |
| PFKFB4 | −1.30 | Protein kinase N1 |
| FDFT1 | −1.30 | Farnesyl-diphosphate farnesyltransferase 1 |
| SQSTM1 | −1.29 | Sequestosome 1 |
| STARD4 | −1.26 | Star related lipid transfer domain containing 4 |
| CYP51A1 | −1.25 | Cytochrome P450 family 51 subfamily A member 1 |
| DNHD1 | −1.21 | Dynein heavy chain domain 1 |
| MVD | −1.20 | Mevalonate diphosphate decarboxylase |
| C2 | −1.14 | Heterogeneous nuclear ribonucleoprotein C |
| PAK1 | −1.14 | PAK1 interacting protein 1 |
| RPS9 | −1.14 | Ribosomal protein S9 |
| RBFOX2 | −1.14 | RNA binding fox-1 homolog 2 |
| POLR3E | −1.12 | RNA polymerase III subunit E |
| SLC39A10 | −1.10 | Solute carrier family 39 member 10 |
| TRNH | −1.08 | Trna-His |
| LOC729973 | −1.08 | Uncharacterized LOC729973 |
| MYEOV | −1.08 | Myeloma overexpressed |
| PCSK9 | −1.08 | 6-phosphofructo-2-kinase/fructose-2,6-biphosphatase 4 |
| C5orf51 | −1.06 | RAB7A interacting MON1-CCZ1 complex subunit 1 |
| RDH11 | −1.06 | Retinol dehydrogenase 11 |
| RRN3 | −1.05 | RRN3 homolog, RNA polymerase I transcription factor |
| HSD17B7 | −1.05 | Hydroxysteroid 17-beta dehydrogenase 7 |

### 3.4. Effects of CA on the Signaling Pathways of Lipid Metabolism

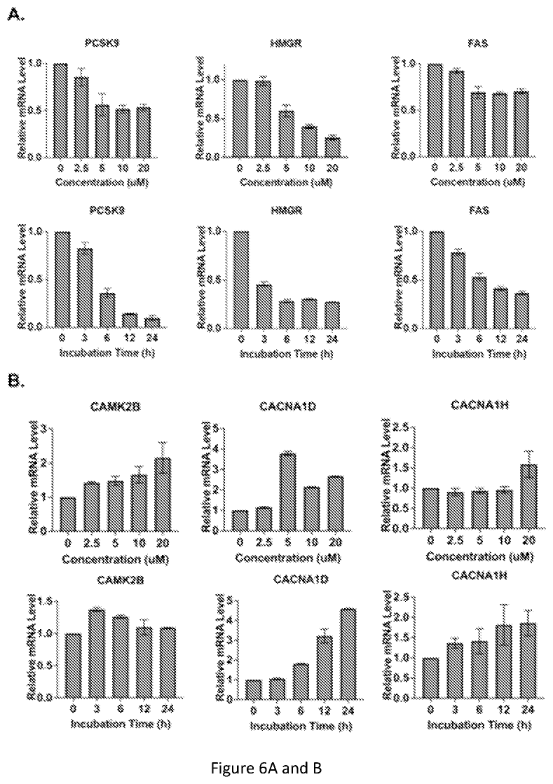

RT-qPCR were used to confirm the down regulation of selected genes involved in lipid metabolism. HepG-2 cells were treated with 0, 2.5, 5, 10, and 20 μM CA for 6 hours, and mRNA levels of key genes involved in lipid metabolism quantitated by RT-qPCR. The results showed that the levels of mRNA encoding PCSK9, HMGR, and FAS were decreased. The decreases in gene expression were concentration and the time dependent as shown in FIG. 6A. Previous reports have shown that the calcium signaling pathway regulates lipid and energy metabolism by down regulating gene expression. The expression of key genes involved in calcium signaling pathway including: CACNA1D (calcium voltage-gated channel subunit alpha1 D), CACNA1H (calcium voltage-gated channel subunit alpha1 H) (encoding for calcium voltage-gated channel subunits), and CAMK2B (Calcium/Calmodulin Dependent Protein Kinase II Beta) were quantitated by RT-qPCR. The results showed that all three genes were up-regulated in a concentration and time dependent manner (FIG. 6B). The results indicate that CA may decrease lipid biosynthesis by upregulation of the calcium-AMPK signaling pathway.

### 3.5. Effects of CA on the Lipid Accumulation in Human Hepatocytes

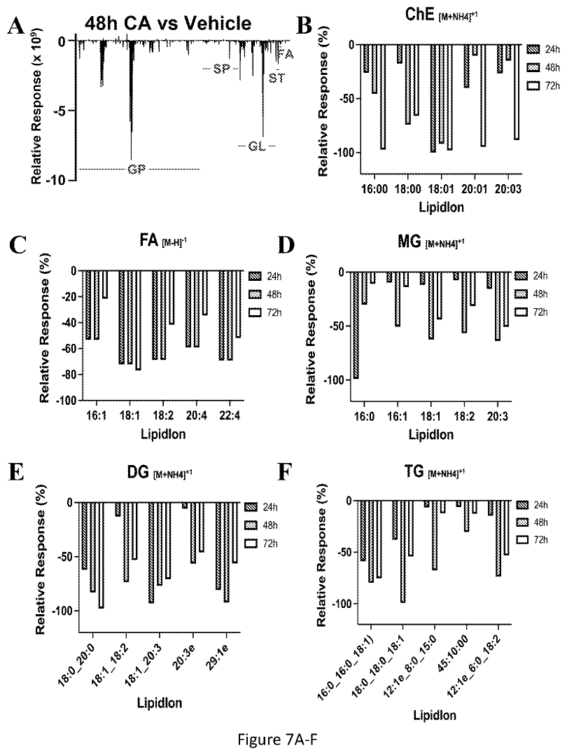

Both WGBS and RNA sequencing results showed that CA may play an important role in lipid metabolism in hepatocytes. To examine the lipid levels in hepatocytes, HepG-2 cells were cultured in HG medium for 72 hours, followed by treatment with 20 μM CA for 24, 48, and 72 hours. Total lipids were measured by untargeted lipidomics assay. The results showed that CA significantly decreased lipid levels, including glycerophospholipids (GP), sphingolipids (SP), glycerolipids (GL), sterol lipid (ST), and fatty acids (FA). The relative decrease levels at 48 hours are shown in FIG. 7A; the major decreased lipids were GP. Further analysis for each lipid group showed that the average decreased percentage of ChE (cholesterol ester) at 48 hours were 47%, the major lipids decreased lipids are shown in FIG. 7B; 65% for FA (FIG. 7C), 53% for MG (Monoglycerides) (FIG. 7D), 76% for DG (Diglycerides) (FIG. 7E) and 70% for TG (Triglycerides) (FIG. 7F). The raw data from lipidomic assay from HepG-2 cells treated with CA for 48 hours are shown in Table S6. The results indicate that CA decreases lipid accumulation via DNA 5 mCpG demethylation, upregulation of key genes involved in calcium-AMPK signaling pathway, and subsequent down-regulation of gene expressions involved in lipid biosynthesis.

### 4. Discussion

CA, 25HC, and 27HC are synthesized by CYP27A1 in mitochondria. The present study reports that CA is a possible unique epigenetic regulator of gene expression in HepG-2 cells. In contrast with 25HC and 27HC, which are potent activators of DNMT1 and silence many genes but do not have any effect on the DNMT3a and DNMT3b enzymatic activities, CA is a potent inhibitor of DNMT3a/b (FIG. 1). The increase of CA levels in nuclei, where DNMT1 and DNMT3b are co-localized, is consistent with its enzyme kinetic results and with its physiological effects on gene regulation. CA is structurally different from sulfated oxysterols that inhibit both DNMT3a/b and DNMT1. The unique chemical structure indicates that CA plays a different role from sulfated oxysterols in regulating gene expression.

25HC and 27HC are endogenous LXR ligands and play important roles in lipid metabolism, inflammatory responses, and cell survival. Recent reports have shown that 25HC and 27HC serve as epigenetic regulators as endogenous activators of DNMT1. High glucose levels induce lipid accumulation in hepatocytes via generating endogenous 25HC and increasing promotor DNA CpG methylation, subsequently silencing key genes regulated by the MAPK-ERK and calcium-AMPK signaling pathways. CYP27A catalyzes oxidations of cholesterol in mitochondria and produces 25HC and 27HC. Further oxidation of 27HC by CYP27A generates CA. The present study shows that CA appears different from 25HC and 27HC in regulating DNMTs: CA up-regulates calcium-AMPK signaling pathways and significantly decreases the expression of key genes; including PSCK9, HMGR, ACC-1, and FAS, which are involved in cholesterol, fatty acid, and triglyceride biosynthesis. The results of the current study indicate that CA may play a preventative role in the development of fatty liver diseases. The regulatory mechanism of CA biosynthesis is unknown. A recent report shows that insulin-resistance dysregulates CYP7B1, and substantially increases the CA levels in liver tissue in mouse models NAFLD, suggesting that CYP7B1 may be a key enzyme in regulating CA levels in vivo.

Unlike 25HC3S and 27HC3S which inhibit DNMT1 and DNMT3a/b, CA activates DNMT1 at the low concentration and inactivate DNMT3a/b. Interestingly, like 25HC3S and 27HC3S, CA suppresses lipid biosynthesis and decreases lipid accumulation in hepatocytes but does not affect cell proliferation or apoptosis. The current results imply that DNMT1 may be responsible for regulating blocks of genes involved in cell proliferation and cell death, and DNMT3a/b may regulate genes involved in lipid metabolism. However, detailed mechanisms of how DNMTs regulate different genes is currently unknown.

25HC, 27HC, CA, and other oxysterols have been reported as endogenous LXR ligands. Whether these sterol metabolites activate LXRs or LXRs serve as a transporters, delivering their ligands into nuclei, where the ligands regulate epigenomic modification by activating/inactivating epigenetic regulators such as DNMTs, has not been investigated. Recent publications have reported that several cholesterol metabolites including oxysterols, and oxysterol sulfates directly activate or inactivate DNMTs in the nuclei and play opposite role in the gene expression. Therefore, it is possible that LXRs may only deliver these molecules into the nuclei, where they regulate gene expression of physiologically linked pathways. The regulation of gene expression by oxysterols through activating/inactivating DNMT enzymatic activity may have an amplifying cascade effect on gene regulation. A recent publication shows that high glucose (HG) levels in culture medium induce lipid accumulation in hepatocytes via epigenetic regulation by 25HC and 27HC.

The present study shows that addition of CA reverses HG-induced lipid accumulation. Based on these results, we propose a new regulatory pathway, which may play an important role in the prevention of NAFLD and metabolic syndrome. When cells are incubated in high glucose, sugar consumption will increase intracellular 25HC and 27HC, which in turn will activate DNMT1, resulting in increase of lipid biosynthesis and lipid accumulation in cells. When intracellular levels increase, CA competes with 25HC and 27HC binding to LXR to enter the nucleus, where CA inhibits DNMT3a/3b and subsequently decreases lipid biosynthesis and lipid accumulation (FIG. 8). Thus, CA has potential to serve as a new biomedicine for prevention and treatment of NAFLD.

### Example 2. Cholestenoic Acid (CA; 33-Hydroxy-5-Cholestenoic Acid) as an Epigenetic Regulator for Treatment of Hepatocellular Carcinoma

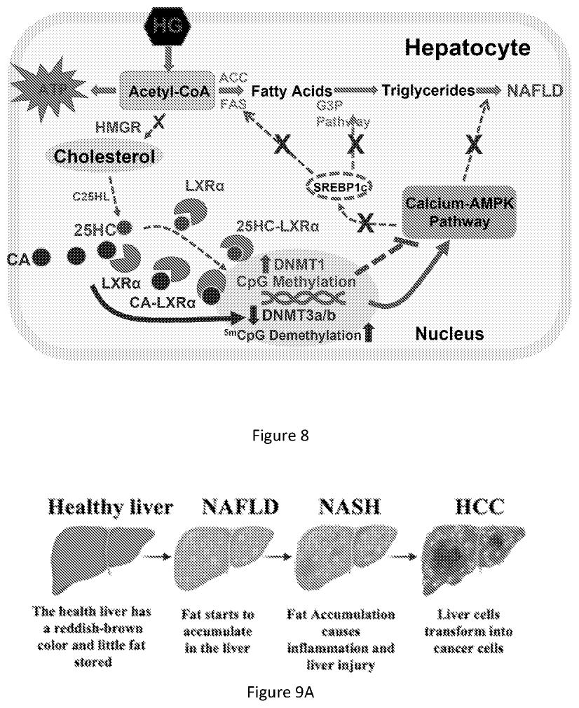

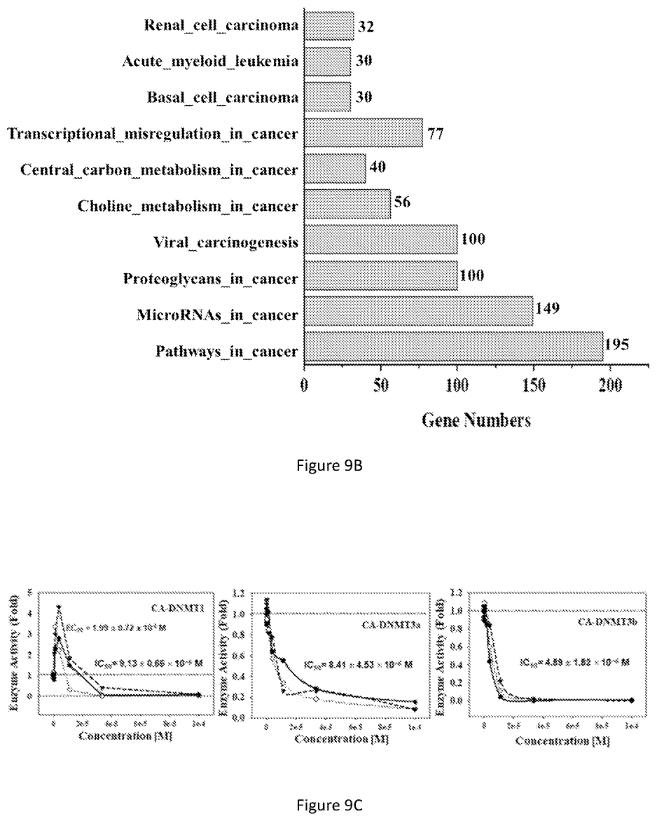

Hepatocellular carcinoma (HCC) is the third leading cause of cancer deaths worldwide, with a relative 5-year survival rate of approximately 18%. The similarity between incidence and mortality (830,000 deaths per year) underlines the dismal prognosis associated with this disease, the progression of which is illustrated in FIG. 9A. The therapy strategies for this disease, for example liver transplantation for early stages, surgical resection, radiofrequency ablation and trans arterial chemoembolization and broad-spectrum tyrosine kinase inhibitors (TKIs) for advanced HCC, provide nominal extension in the survival curve, cause broad spectrum toxic side effects, and patients eventually develop therapy resistance. Therefore, there is a dire need for the development of an efficient and safe therapy for HCC.

Recent studies have identified higher DNA methylation levels in HCC compared with normal cells. DNA methylation is an epigenomic modification that controls gene expression (FIG. 9B). It has been reported that 6 cancer biomarker genes, including TL, DUSP1, EOMES, ESM1, NFKBIA and SOCS2, were down-regulated with high methylation levels in HCC.

Our studies have shown that cholestenoic acid (CA) significantly inhibited DNA methyltransferases 1/3a/3b (DNMT 1/3a/3b) activities with IC₅₀=9.13×10⁻⁶ M, IC₅₀=8.41×10⁻⁶ M and IC₅₀=4.89×10⁻⁶ M, respectively (FIGS. 9B and C). Further, CA stimulated demethylation of 5 mCpG to CpG in the promoter regions of over 7,000 genes in HepG-2 cells (a human hepatoblastoma cell line), and regulated expression of genes involved in cancer growth and death related pathways.

Further study has shown that CA induces HepG-2 cell death but not the death of normal primary human hepatocytes (PHH) as shown in FIGS. 10A and B.

Based on these findings, CA is an ideal molecule to serve as an inhibitor of DNMTs for HCC therapy.

### Example 3. Animal Studies of the Impact of CA on Development of Nonalcoholic Fatty Liver Disease (NAFLD)

The animal studies were approved by the Institutional Animal Care and Use Committee of McGuire Veterans Affairs Medical Center and conducted in accordance with the Declaration of Helsinki, the Guide for the Care and Use of Laboratory Animals, and all applicable regulations. In this study, a western diet-induced nonalcoholic fatty liver disease (NAFLD) mice model was used. To create the model, eight-week-old C57BL/6J female mice were purchased from the Jackson Laboratory and fed a western diet (TD.88137, Envigo) along with high glucose/fructose water (WDSW) containing 23.1 g/L fructose and 18.9 g/L glucose for 12 weeks. After establishing the model, the mice were separated into three groups based on their weight. For the control mice in each group, they received intravenous injection (IV) with vehicle (DMSO). In contrast, the treatment group mice were intravenously injected with 10 mg/kg of CA (dissolved in DMSO) with a total volume of less than 100 μl. During the treatment period, injections were administered every two days. All mice were housed under identical conditions in an aseptic facility with a 12-hour light/12-hour dark cycle and provided with free access to water and food (WDSW). Before scarification, the mice fasted overnight. Blood samples were collected, and the serum enzymatic activities of alkaline phosphatase (ALK), alanine aminotransferase (ALT), and aspartate aminotransferase (AST) were measured in the clinical laboratory at McGuire Veterans Affairs Medical Center. These enzymes constitute a routine liver function test and represent markers for liver inflammation, which is typically elevated in individuals with a fatty liver (e.g., NAFLD).

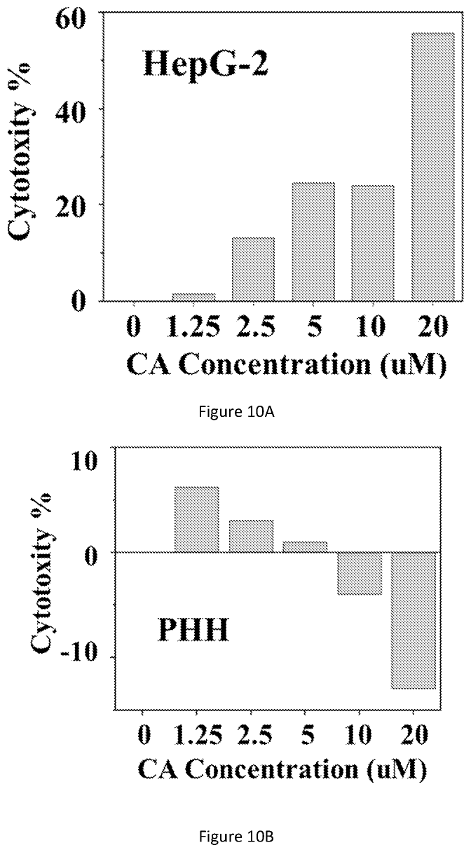

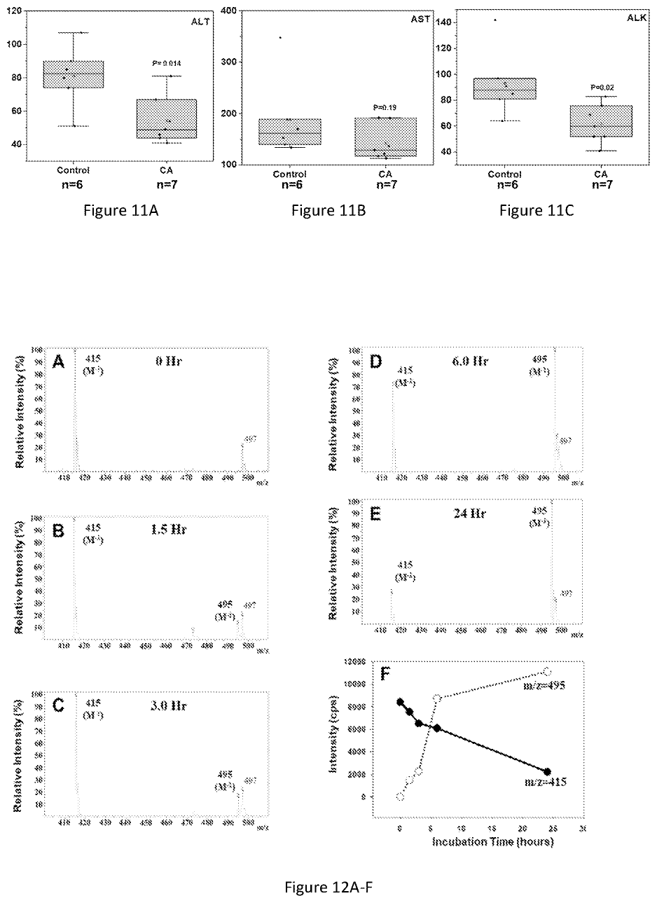

The results are presented in FIG. 11A-C.

In conclusion, this study suggests that CA as a unique endogenous epigenetic regulator decreases lipid accumulation via epigenomic modification and regulation of key gene expression involved in calcium-AMPK signaling pathway. The results indicate that CA has potential as a therapeutic target for lipid accumulation-associated diseases.

### Example 4. Novel Secreted Regulatory Cholestenoic Acid Derivative, 33-Sulfate-5-Cholestenoic Acid (CA3S), as Biomedicine for Therapy of Inflammatory Response-Associated Diseases

Mitochondrial oxysterols, including cholestenoic acid (CA), 25-hydroxycholesterol (25HC), and 27-hydroxycholesterol (27HC), are potent regulators involved in important biological events such as lipid metabolism and inflammatory responses. However, their intracellular catabolic pathways have not been fully explored. In this study, we investigated the metabolic pathways of these oxysterols and their roles in the communication between hepatocytes and macrophages. Using LC-MS-MS analysis, we traced the metabolites of these oxysterols and found that a novel molecular ion (m/z) 495 appeared at 1.5 hr and reached a maximum (90%) at 24 hr when CA was added to media culturing Hep G2 cells (FIG. 12A-F). Daughter spectra showed that m/z 80 was attached to m/z 415 (CA) and using isotopic (five deuterium) labeled ^{d5}-CA confirmed that m/z 495 was a derivative of m/z 415.

CA3S was synthesized as follows: A mixture of cholestenoic acid (13 mg, 0.03 mmol) and triethylamine-sulfur trioxide (7 mg, 0.038 mmol) was dissolved in dry pyridine (0.6 ml) and was stirred at 50° C. for 2 hours. The solvents were evaporated at 40° C. under nitrogen stream, and the syrup was added into 5 ml of 50% acetonitrile (loading buffer). The products were applied to a 6 cc of Oasis cartridges (Waters), which had been primed by methanol (15 mL) and water (15 mL). The cartridge was successively washed with the loading buffer (15 mL), water (15 mL), methanol (15 mL), 50% methanol (15 mL), 5% ammonia hydroxide in 10% methanol (15 mL), and 5% ammonia hydroxide in 50% methanol (15 mL). The retained CA3S was eluted with 5% ammonia hydroxide in 80% methanol (10 mL). After dilution with 10 times volume of acetonitrile, the solvents were evaporated to dryness under nitrogen stream, and the CA3S was obtained as white powder form. Yield was ˜90%.

We compared the synthesized compound and confirmed that m/z 495 is 3J-hydroxy-5-cholestenoic acid 3-sulfate (CA3S) (FIGS. 13A and B).

When 27HC was added, ⅔ of the compound was converted to CA3S and ⅓ to 27HC3S. Both were released into the media. With the addition of 25HC, that compound was converted to 25HC3S and only ⅓ was released into the media, and the rest remained in the cells and nuclei.

Based on our results, we conclude that CA is derived from 27HC and further sulfated to CA3S, which acts as a secretion regulator for the regulation of inflammatory responses. These findings provide insight into the intracellular catabolic pathways of these oxysterols and their roles in cellular communication.

CA3S is thus a derivative of cholestenoic acid which is secreted from hepatocytes and acts on macrophages. Further experiments showed that CA3S has potent cholesterol and triglyceride lowering and anti-inflammatory properties. CA3S has been shown to be able to suppress inflammatory responses by suppressing pro-inflammation gene expression. The decreases in pro-inflammation cytokine gene expression can lead to suppressed inflammatory responses. Thus, CA3S is useful for treating diseases associated with inflammatory responses, such as sepsis, metabolic associated fatty liver diseases (lipotoxicity), and atherosclerosis.

Further experiments showed that CA3S suppresses key gene expressions involved in LPS induced inflammatory responses in macrophages. Human THP-1 were plated in 6 well plates with RPMI 1640 media (10% FBS) and induced to be macrophages by 100 nM PMA for 3 days at 37° C. in a 0.5% CO₂ incubator. The media was then changed to fresh RPMI 1640 (serum free), the cells were treated with 20 uM CA3S in PG for 30 min, and then the cells were stimulated with 2 ug/ml LPS for 6 hours. Total mRNA was extracted by the Promega SV total RNA isolation system (Promega, Madison, WI, USA) and 1 ug of RNA were converted to cDNA with a Reverse Transcription kit (Qiagen, Hilden, Germany). IL-1A, IL-1B, IL-6, IL-8, COX-2, NFKB and TNFα gene expressions were determined by Real-time RT-PCR that was performed using SYBR Green as the indicator on ABI 7500 Fast Real-Time PCR System (Applied Biosystems, Foster City, CA).

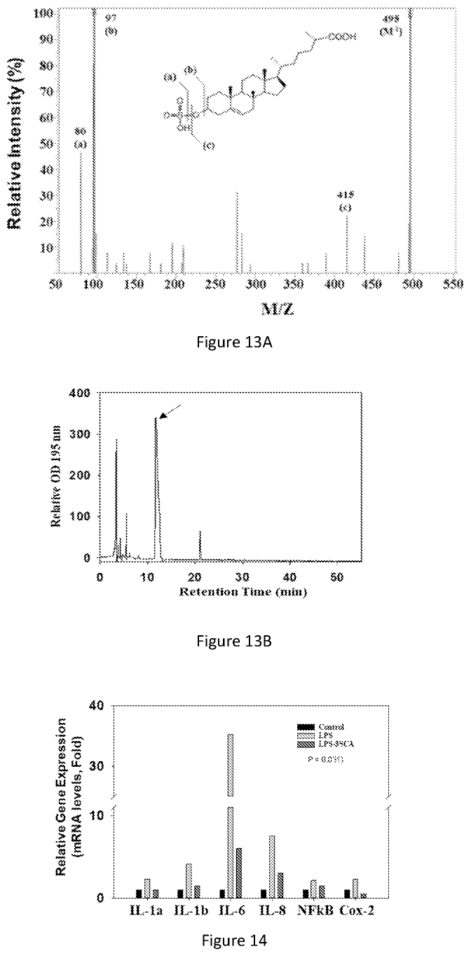

The results are presented in FIG. 14. As can be seen, CA3S decreases IL-6 by 6-fold, and COX-2 by 5-fold. This property of decreasing anti-inflammatory responses will benefit recovery from many fatal diseases, such as sepsis shock, metabolic disorder fatty liver diseases (MAFLD), and atherosclerosis. These diseases are caused by systematic inflammatory responses, which currently has no approved therapy.

Additional investigations showed that the addition of CA3S significantly suppressed inflammatory responses via G-coupling signaling pathway in macrophages (not shown).

### Example 5. Animal Studies of the Impact of CA3S on Development of Nonalcoholic Fatty Liver Disease (NAFLD)

The animal studies were approved by the Institutional Animal Care and Use Committee of McGuire Veterans Affairs Medical Center and conducted in accordance with the Declaration of Helsinki, the Guide for the Care and Use of Laboratory Animals, and all applicable regulations. In this study, a western diet-induced nonalcoholic fatty liver disease (NAFLD) mice model was used. To create the model, eight-week-old C57BL/6J female mice were purchased from the Jackson Laboratory and fed a western diet (TD.88137, Envigo) along with high glucose/fructose water (WDSW) containing 23.1 g/L fructose and 18.9 g/L glucose for 12 weeks. After establishing the model, the mice were separated into three groups based on their weight. For the control mice in each group, they received intravenous injection (IV) with vehicle (DMSO). In contrast, the treatment group mice were intravenously injected with 10 mg/kg of CA3S (dissolved in DMSO) with a total volume of less than 100 ul. During the treatment period, injections were administered every two days. All mice were housed under identical conditions in an aseptic facility with a 12-hour light/12-hour dark cycle and provided with free access to water and food (WDSW). Before scarification, the mice fasted overnight.

Blood samples were collected, and the serum enzymatic activities of alkaline phosphatase (ALK), alanine aminotransferase (ALT), and aspartate aminotransferase (AST) were measured in the clinical laboratory at McGuire Veterans Affairs Medical Center. These enzymes constitute a routine liver function test and represent markers for liver inflammation, which is typically elevated in individuals with a fatty liver (e.g., NAFLD).

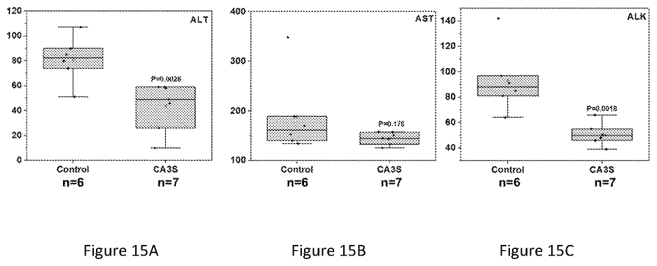

The results are presented in FIG. 15A-C. In conclusion, this study confirms that CA3S is a unique endogenous epigenetic regulator that can be used successfully to prevent and/or treat NAFLD.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A compound which is 3 β-hydroxy-5-cholestenoic acid 3-sulfate (CA3S) or salts or solvates thereof.

2. A pharmaceutical composition comprising the compound of claim 1 and a pharmaceutically acceptable carrier, the compound being dissolved or distributed in the carrier, wherein the compound comprises 1-99% of the composition.

3. The pharmaceutical composition of claim 2, wherein the composition is formulated in unit dosage form.

4. The pharmaceutical composition of claim 2, wherein the composition is in solid form.

5. The pharmaceutical composition of claim 4, wherein:
the composition is in the form of a powder, a tablet, a capsule or a lozenge; or
the composition comprises the compound in freeze-dried form together with a bulking agent.

6. The pharmaceutical composition of claim 2, wherein the carrier is a liquid.

7. The pharmaceutical composition of claim 6, wherein:
the compound is solubilized in the liquid or dispersed in the liquid; and/or
the liquid is aqueous; and/or
the liquid is sterile water for injections or phosphate-buffered saline; and/or
the composition is in a sealed vial, ampoule, syringe or bag.

8. A method of treating a subject, which method comprises administration to the subject of an effective amount of a compound as defined in claim 1, wherein the method is selected from: a method for treating sepsis in a subject in need thereof; a method for treating metabolic associated fatty liver diseases in a subject in need thereof; a method for reducing lipids in a subject in need thereof; a method of reducing cholesterol and lipid biosynthesis in a subject in need thereof; a method of reducing inflammation in a subject in need thereof; a method of treating diabetes in a subject in need thereof; a method of treating hyperlipidemia in a subject in need thereof; a method of treating atherosclerosis in a subject in need thereof; a method of treating fatty liver disease in a subject in need thereof; and a method of treating inflammatory disease in a subject in need thereof.

9. The method of claim 8 wherein:
the compound is administered in an amount ranging from 0.1 mg/kg to 100 mg/kg based on body mass of the subject, or the compound is administered in an amount ranging from 1 mg/kg to 10 mg/kg, based on body mass of the subject; and/or
the administration comprises at least one of oral administration, enteric administration, sublingual administration, transdermal administration, intravenous administration, peritoneal administration, parenteral administration, administration by injection, subcutaneous injection, and intramuscular injection.

10. A method of lowering lipids in a subject in need thereof, comprising
administering to the subject a therapeutically effective amount of at least one of 3β-hydroxy-5-cholestenoic acid (CA) and CA3S.

11. The method of claim 10, wherein the lipids are cholesterol and/or triglycerides.

12. The method of claim 10, wherein the therapeutically effective amount is from 1-100 mg/kg of body weight.

13. A method of preventing or treating an inflammatory disease or condition in a subject in need thereof, comprising
administering to the subject a therapeutically effective amount of at least one of CA and CA3S.

14. The method of claim 13, wherein the inflammatory disease or condition is sepsis, metabolic associated fatty liver diseases, or atherosclerosis.

15. The method of claim 13, wherein the therapeutically effective amount is from 1-100 mg/kg of body weight.

16. A method of preventing or treating non-alcoholic fatty liver disease (NAFLD) in a subject in need thereof, comprising
administering to the subject a therapeutically effective amount of at least one of CA and CA3S.

17. The method of claim 16, wherein the therapeutically effective amount is from 1-100 mg/kg of body weight.

18. A method of treating cancer in a subject in need thereof, comprising
administering to the subject a therapeutically effective amount of CA.

19. The method of claim 18, wherein the cancer is hepatocellular carcinoma.
